(19) United States
(12) Patent Application Publication
(10) Pub. No.: US 2005/0106124 A1
(43) Pub. Date: May 19, 2005

(54) Therapeutic applications of thrombomodulin gene via viral and non-viral vectors

(76) Inventors: Lakshman R. Sehgal, Glenview, IL (US); Jonathan Wong, Chicago, IL (US); Prem Seth, Glenview, IL (US)

Correspondence Address: Supervisor, Patent Prosecution Services;PIPER RUDNICK LLP, 1200 Nineteenth Street, N.W., Washington, DC, 20036-2412, US

(21) Appl. No.: 10/785,156

(22) Filed: Feb. 25, 2004

Related U.S. Application Data
(60) Provisional application No. 60/449,408, filed on Feb. 25, 2003.

(51) Int. Cl. A61K 48/00; C12N 15/861

(52) U.S. Cl. 424/93.2; 435/456

(57) ABSTRACT

The present invention relates to methods and compositions for treating thrombotic diseases using gene delivery technologies. In one embodiment, a therapeutically effective amount of a TM gene product is expressed in a TM-deficient mammal using a viral or non viral vector. In another embodiment, the vector-mediated in vivo TM gene expression is used for the treatment of atherosclerotic cardiovascular disease, pulmonary hypertension, acute inflammatory diseases, end-stage renal failure disease, or Alzheimer disease. In yet another embodiment, a vector carrying a TM inhibitory polynucleotide in which the vector is introduced into a mammal to reduce the TM activity or TM gene expression in vivo.

This application claims priority from U.S. Provisional Application Ser. No. 60/449,408 filed Feb. 25, 2003. The entirety of that provisional application is incorporated herein by reference.

### FIELD OF THE INVENTION

The present invention is directed to methods and compositions of the treatment of thrombotic diseases and, in particular, to the treatment of atherosclerotic cardiovascular disease, pulmonary hypertension, acute inflammatory diseases, end-stage renal failure disease, and Alzheimer disease by modulating expression of the thrombomodulin gene.

### BACKGROUND OF THE INVENTION

Thrombomodulin (TM) is an integral membrane glycoprotein expressed on the surface of endothelial cells. It is a high affinity thrombin receptor that converts thrombin into a protein C activator. Activated protein C then functions as an anticoagulant by inactivating two regulatory proteins of the clotting system, namely factors Va and VI[I1a. The latter two proteins are essential for the function of two of the coagulation proteases, namely factors IXa and Xa. TM, thus, plays an active role in blood clot formation in vivo and can function as a direct or indirect anticoagulant.

TM is a single chain protein composed of 5 distinct domains. A short cytoplasmic domain containing a free cysteine is located at the COOH-terminal end and is joined by a membrane spanning region to an o-glycosylation rich domain. The latter is followed by an epidermal growth factor (EGF) homology region and the NH2-terminal hydrophobic domain. The EGF homology region contains 6 EGF like domains and contains the binding sites for both thrombin and protein C.

TM is also prevalent in other cell types includes keratinocytes, osteoblasts, macrophages. In these cells/tissues, TM is involved in the differentiation and inflammation. Abnormal TM function is also associated with many diseases. For example, abnormal TM in the endothelial cells contribute to myocardial infarction (MI), stroke and the development of atherosclerotic plaque. In other diseases, natural TM is missing, deficient or simply cleaved into soluble form. Therefore, modulation of in vivo TM expression is desirable in these clinical scenarios.

### SUMMARY OF THE INVENTION

The present invention provides a method for treating a thrombotic disease in a mammal comprising administering to the mammal a therapeutically effective amount of a pharmaceutical composition comprising a viral or a non-viral vector, wherein the viral or non-viral vector comprises an isolated nucleotide sequence encoding thrombomodulin and its variant. The present invention also provides a method for treating a thrombotic disease in a mammal comprising administering to the mammal an effective amount of thrombomodulin-producing cells, wherein said thrombomodulin-producing cells are generated by introducing an isolated polynucleotide encoding an amino acid sequence of thrombomodulin or its variant into cultured cells.

The vector- or cell-mediated in vivo TM gene expression may used for the treatment of any thrombomodulin-related diseases, such as atherosclerotic cardiovascular disease, pulmonary hypertension, acute inflammatory diseases, end-stage renal failure disease, or Alzheimer disease. The present invention further provides a vector carrying an isolated polynucleotide in which the vector is introduced into a mammal to reduce the TM activity or TM gene expression in vivo.

### BRIEF DESCRIPTION OF THE FIGURES

FIG. 1 is a schematic drawing of an embodiment of the backbone shuttle vector of the present invention.

FIG. 2 is the DNA sequence (SEQ ID NO: 1) of the gutless backbone shuttle vector.

FIG. 3 is the full length amino acid sequence (SEQ ID NO:2) of human thrombomodulin.

FIG. 4 is the full length DNA sequence (SEQ ID NO:3) encoding human thrombomodulin.

FIG. 5 is the DNA sequence (SEQ ID NO:4) of the expression cassette encoding human thrombomodulin.

FIG. 6 is the DNA sequence (SEQ ID NO:5) of the CMV promoter of the expression cassette encoding the human thrombomodulin.

FIG. 7 is the cDNA (SEQ ID NO:6) of the human thrombomodulin gene.

### DETAILED DESCRIPTION OF THE INVENTION

The primary objective of the present invention is to provide methods and compositions for treating diseases or conditions relating to the TM expression. One aspect of the invention relates to the treatment for diseases or conditions associated with reduced TM expression or loss of TM activity. These diseases may be treated by expressing a therapeutically effective amount of the TM protein in vivo using a viral or a non-viral vector. Another aspect of the invention relates to the treatment for diseases associated with enhanced TM expression. Under these conditions, TM gene express or TM activity may be inhibited by the in vivo expression of a TM inhibitory polynucleotide using a gene expression vector.

The practice of the present invention will employ, unless other wise indicated, conventional methods of histology, virology, microbiology, immunology, and molecular biology within the skill of the art. Such techniques are explained fully in the literature. All publications, patents and patent applications cited herein, whether supra or infra, are hereby incorporated by reference in their entirety.

Definitions

In describing the present invention, the following terms will be employed, and are intended to be defined as indicated below.

“Gene transfer” or “gene delivery” refers to methods or systems for reliably introducing a particular nucleotide sequence (e.g., DNA) into targeted cells. The introduced nucleotide sequences may persist in vivo in episomal forms or integrate into the genome of the target cells. Gene transfer provides a unique approach for the treatment of acquired and inherited diseases, and a number of systems have been developed in the art for gene transfer into mammalian cells. See, e.g., U.S. Pat. No. 5,399,346.

As used herein, the term “therapeutically effective amount” refers to a level of transfection which brings about at least partially a desired therapeutic or prophylactic effect in an organ or tissue infected by the method of the present invention. The transfection with a therapeutically effective amount of the vector carrying genetic material of interest can then result in the modification of the cellular activities, e.g., a change in phenotype, in an organ or a tissue that has been infected by the method of the present invention. In a preferred embodiment, the transfection with an effective amount of the vector carrying genetic material of interest results in modulation of cellular activity in a significant number of cells of an infected organ or a tissue.

A gene transfer “vector” refers to any agent, such as a plasmid, phage, transposon, cosmid, chromosome, liposome, DNA-viral conjugates, RNA/DNA oligonucleotides, virus, bacteria, etc., which is capable of transferring gene sequences into cells. Thus, the term includes cloning and expression vehicles including “naked” expression vectors, as well as viral and non-viral vectors. A vector may be targeted to specific cells by linking a target molecule to the vector. A targeting molecule is any agent that is specific for a cell or tissue type of interest, including for example, a ligand, antibody, sugar, receptor, or other binding molecule. The invention is also intended to include such other forms of vectors which serve equivalent functions and which become known in the art subsequently hereto.

The term “expression control element” or “regulatory element” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a, DNA regulatory sequence that are sufficient for RNA polymerase recognition, binding and transcription initiation. Additionally, a promoter includes sequences that modulate the recognition, binding and transcription initiation activity of RNA polymerase. Such sequences may be cis acting or may be responsive to trans acting factors. Depending upon the nature of the regulation, promoters may be constitutive or regulated. Examples of promoters are SP6, T4, T7, SV40 early promoter, cytomegalovirus (CMV) promoter, mouse mammary tumor virus (MMTV) steroid-inducible promoter, Moloney murine leukemia virus (MMLV) promoter, phosphoglycerate kinase (PGK) promoter, muscle creatine kinase (MCK) promoter, myosin promoter, α-actin promoter and the like.

The term “transduction” denotes the delivery of a DNA molecule to a recipient cell either in vivo or in vitro, via a replication-defective viral vector, such as via a recombinant AAV virus.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as the function to direct the expression thereof. Thus, for example, intervening untranslated yet transcribed sequences can be present between a promoter sequence and the coding sequence and the promoter sequence can still be considered “operably linked” to the coding sequence.

The term “native thrombomodulin” refers to both the natural protein and soluble peptides having the same characteristic biological activity of membrane-bound or detergent solubilized (natural) thrombomodulin. These soluble peptides are also referred to as “wild-type” or “non-mutant” analog peptides. Biological activity is the ability to act as a receptor for thrombin, increase the activation of protein C, or other biological activity associated with native thrombomodulin. Oxidation resistant TM analogs are these soluble peptides that in addition to being soluble contain a specific artificially induced mutation in their amino acid sequence.

“Thrombotic disease” refers to a pathogenic condition in a mammal characterized by the formation of one or more thrombi that are or can be detrimental to the health of the mammal. Examples of the thrombotic diseases include, but are not limited to, atherosclerotic cardiovascular disease, pulmonary hypertension, acute inflammatory disease, end-stage renal failure disease, Alzheimer disease, acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve.

The term “thrombomodulin variant” is a polypeptide that differs from a native thrombomodulin polypeptide in one or more substitutions, deletions, additions and/or insertions, such that the bioactivity of the native thrombomodulin polypeptide is not substantially diminished or enhanced. In other words, the bioactivity of a thrombomodulin variant may be enhanced or diminished by, less than 50%, and preferably less than 20%, relative to the native protein. Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the − and/or C-terminal of the mature protein.

Preferably, a thrombomodulin variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the bioactivity, secondary structure and hydropathic nature of the polypeptide.

Thrombomodulin variants preferably exhibit at least about 70%, more preferably at least about 90% and most preferably at least about 95% sequence homology to the original thrombomodulin polypeptide.

A thrombomodulin variant also include a thrombomodulin polypeptides that is modified from the original thrombomodulin polypeptides by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The present invention also relates to fragments of thrombomodulin. A fragment of thrombomodulin may comprise 5 to 575 consecutive amino acids of thrombomodulin, preferably comprise 20 to 575 consecutive amino acids of thrombomodulin, more preferably comprise 100 to 575 consecutive amino acids of thrombomodulin, and most preferably comprise 200 to 575 consecutive amino acids of thrombomodulin.

In Vivo Thrombomodulin Gene Transfer

The amino acid sequence of human thrombomodulin (SEQ ID NO: 2) and the DNA sequence encoding human thrombomodulin (SEQ ID NO:3) have been reported (Suzuki et al., EMBO J. 6:1891-1897, [1987]). Somatic gene transfer techniques offer a new approach to replace a defective thrombomodulin gene or to modulate in vivo thrombomodulin gene expression. A preferred approach for introducing genetic material encoding a gene product into an organ or a tissue is by use of a gene transfer vector. Commonly used gene transfer vectors include viral vectors and non-viral vectors. In the case of a viral vector, the genetic material encoding thrombomodulin or a thrombomodulin variant is inserted into the viral genome (or a partial viral genome) using molecular cloning techniques well known in the art. The regulatory elements directing the expression of the thrombomodulin or thrombomodulin variant can be included with the genetic material inserted into the viral genome (i.e., operably linked to the gene inserted into the viral genome) or can be provided by the viral genome itself, for example, a retrovirus long terminal repeat (LTR) or an Adeno-associated virus (AAV) inverted terminal repeat (ISR). Transfection of cells with a viral vector has the advantage that molecules encoded within the viral vector, e.g., by a cDNA contained in the viral vector, are expressed efficiently in cells which have taken up viral vector nucleic acid and viral vector systems can be used in vivo. Different viral vectors are described separately in the subsections below.

1. Adenovirus vectors: The genome of an adenovirus can be manipulated such that it encodes and expresses a gene product of interest but is inactivated in terms of its ability to replicate in a normal lyric viral life cycle (Curie, Ann N Y Acad Sci 886:158-171, [1991]). Suitable adenoviral vectors derived from the adenovirus strain Ad type 5 d1324 or other strains of adenovirus (e.g., Ad2, Ad3, Ad7 etc.) are well known to those skilled in the art. Recombinant adenovirus es are advantageous in that they do not require dividing cells to be effective gene delivery vehicles and can be used to infect a wide variety of cell types, including airway epithelium, endothelial cells and muscle cells. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Haj-Ahmand et al., J. Virol. 57:267-273, [1986]). Most replication-defective adenoviral vectors currently in use are deleted for all or parts of the viral E1 and E3 genes but retain as much as 80% of the adenoviral genetic material.

Adenovirus vectors have been successfully tested in a number of animal models (Ragot et al., Nature 361:647-650, [1993]; Howell et al., Hum Gene Ther 9:629-634, [1998]). Nonetheless, the toxicity and immunogenicity remain major hurdles to overcome before the adenoviral vectors can be safely used in humans.

Adenoviral vectors deleted of all viral coding regions (gutless adenoviral vectors) are also described by Kochanek et al., and Chamberlain et al., (U.S. Pat. No. 5,985,846 and U.S. Pat. No. 6,083,750). A new viral backbone shuttle vector was also developed for the construction of gutless adenoviral vectors (U.S. patent application Ser. No. 10/725,013, the entirety of which is incorporated herein by reference).

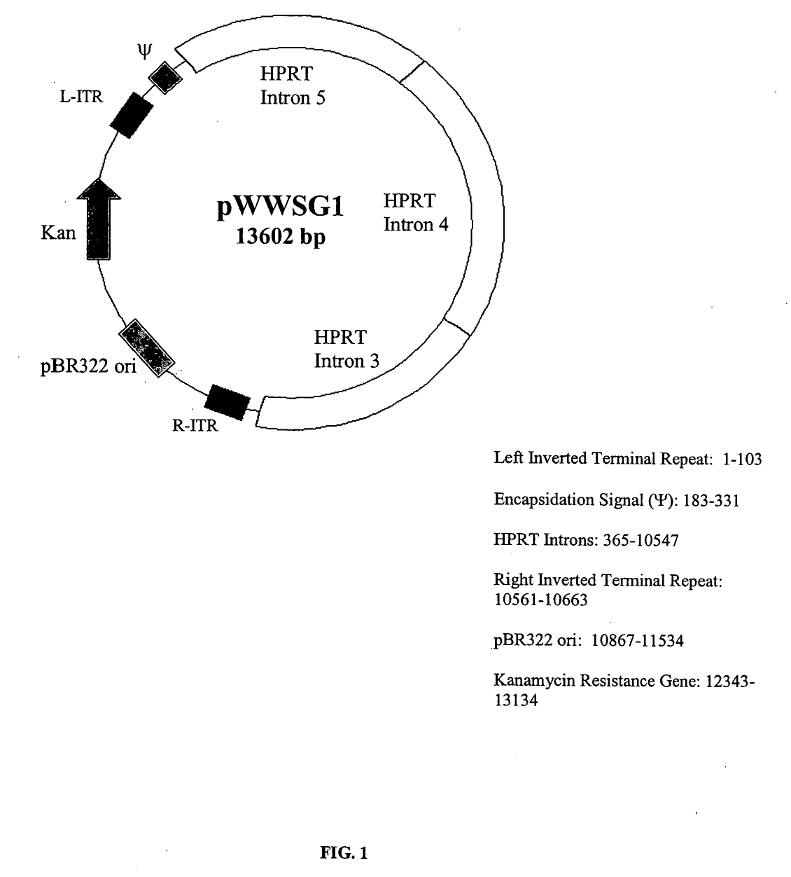

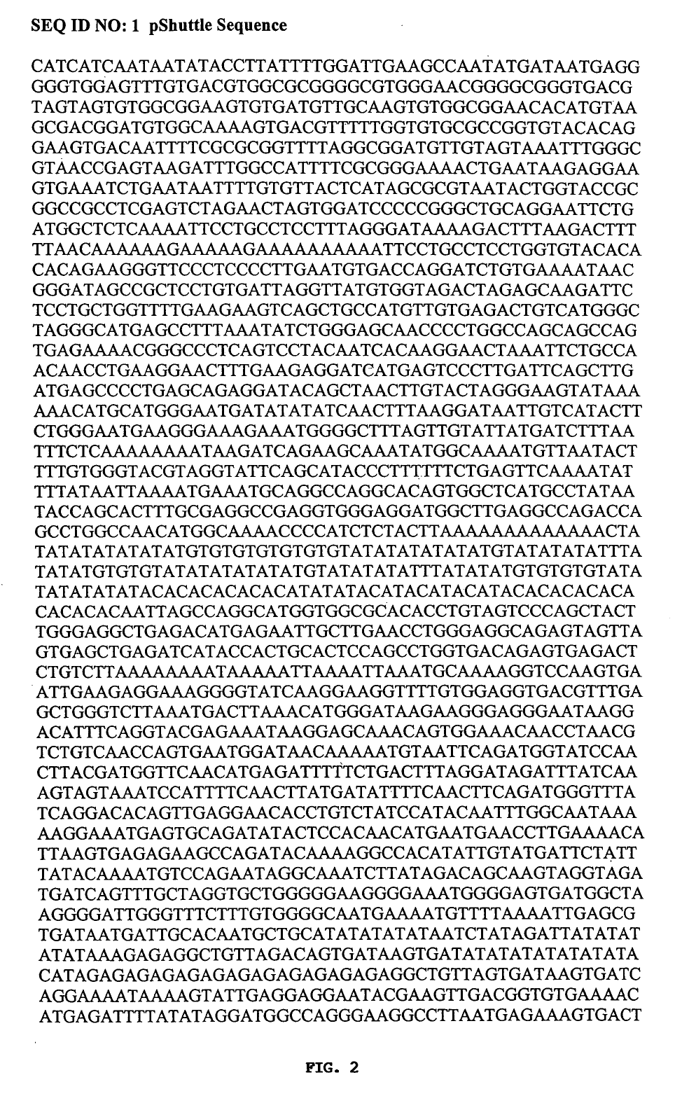

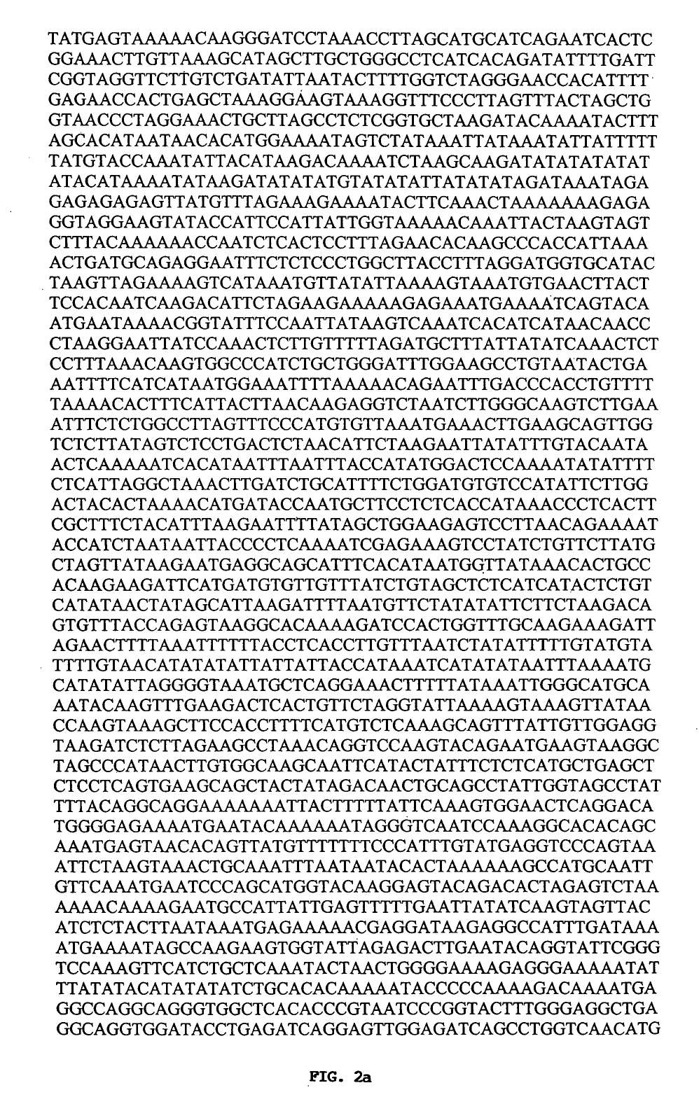

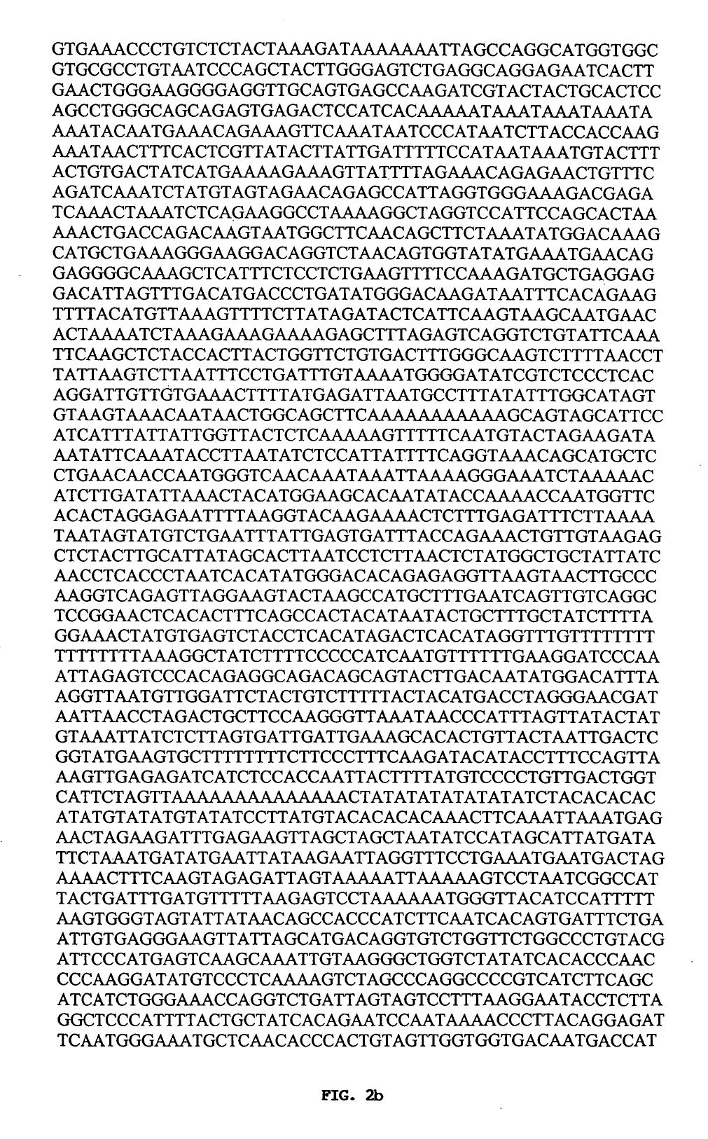

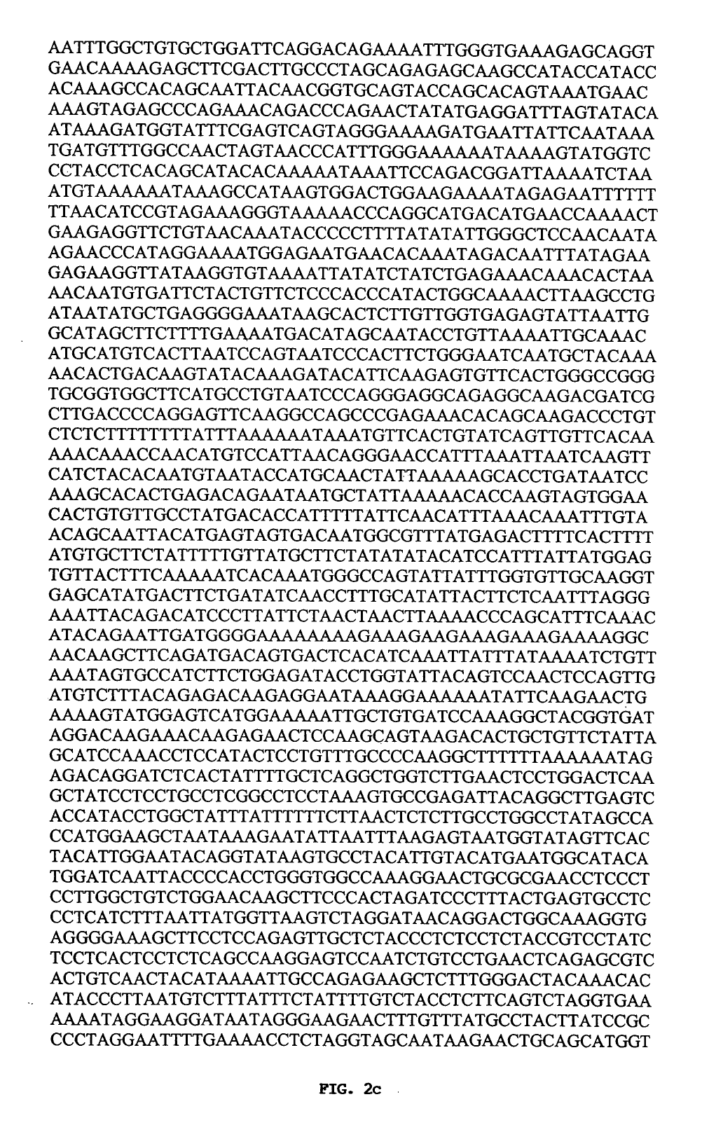

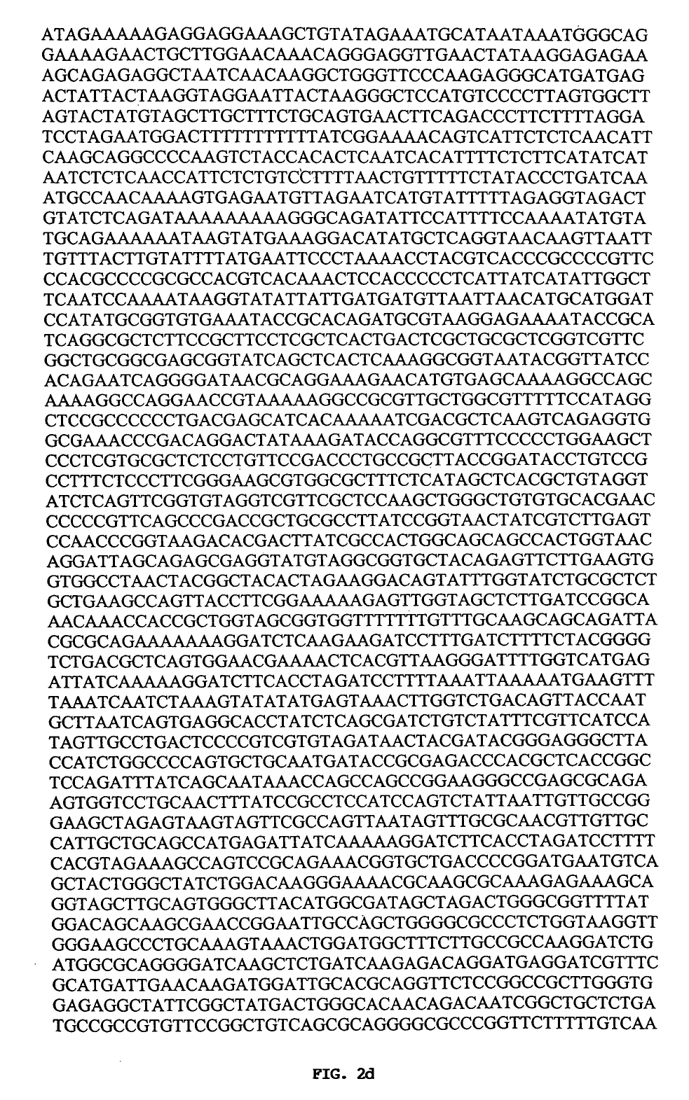

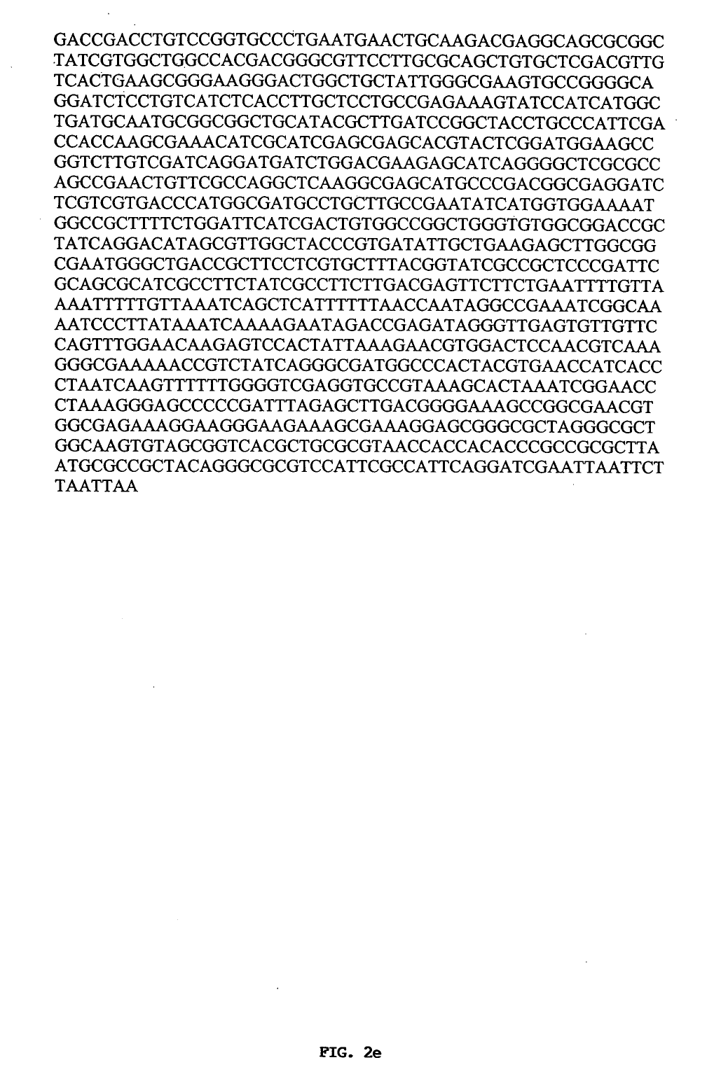

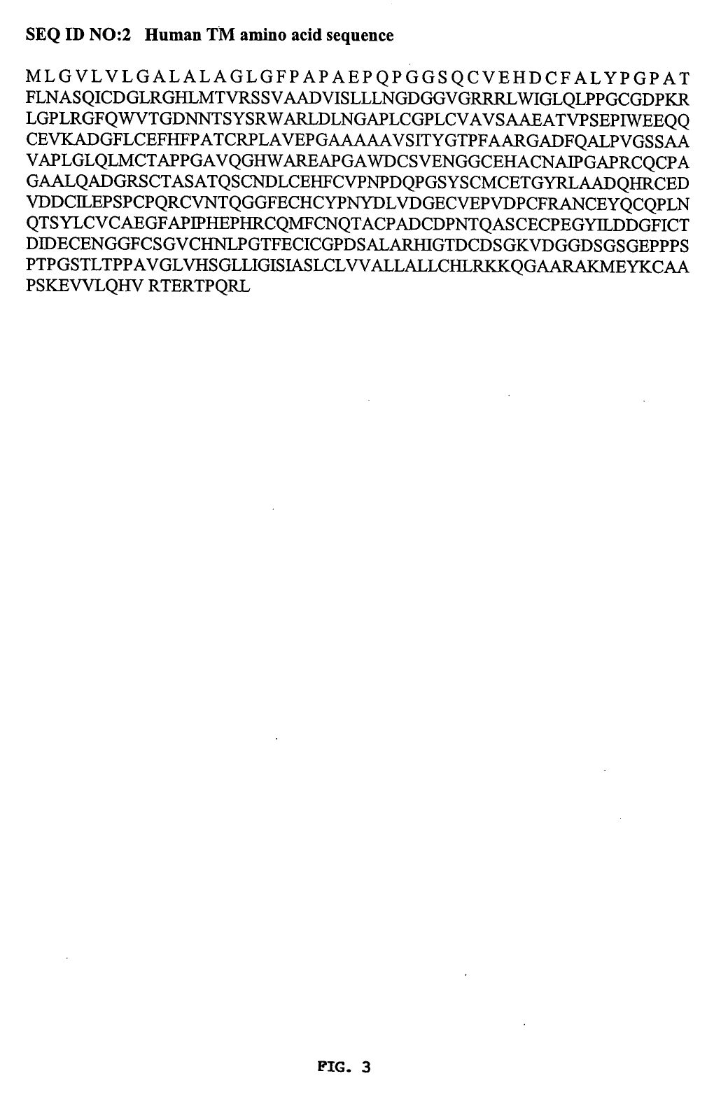

The viral backbone shuttle vector may contain a left and a right inverted terminal repeats of adenovirus, an encapsidation signal (ψ) of adenovirus, a pBR322 replication origin, a kanamycin resistance gene, and a stuffer sequence, which is the hypoxanthine phosphoribosyltransferase (HPRT) intro fragment with an approximately 10 Kb. (FIG. 1).

The “inverted terminal repeats (ITRs) of adenovirus” are short elements located at the 5′ and 3′ termini of the linear Ad genome, respectively and are required for replication of the viral DNA. The left ITR is located between 1-130 bp in the Ad genome (also referred to as 0-0.5 mu). The right ITR is located from about 3,7500 bp to the end of the genome (also referred to as 99.5-100 mu). The two ITRs are inverted repeats of each other. For clarity, the left ITR or 5′ end is used to define the 5′ and 3′ ends of the ITRs. The 5′ end of the left ITR is located at the extreme 5′ end of the linear adenoviral genome; picturing the left ITR as an arrow extending from the 5′ end of the genome, the tail of the 5′ ITR is located at mu 0 and the head of the left ITR is located at about 0.5 mu (further the tail of the left ITR is referred to as the 5′ end of the left ITR and the head of the left ITR is referred to as the 3′ end of the left ITR). The tail of the right or 3′ ITR is located at mu 100 and the head of the right ITR is located at about mu 99.5; the head of the right ITR is referred to as the 5′ end of the right ITR and the tail of the right ITR is referred to as the 3′ end of the right ITR. In the linear Ad genome, the ITRs face each other with the head of each ITR pointing inward toward the bulk of the genome. When arranged in a “tail to tail orientation” the tails of each ITR (which comprise the 5′ end of the left ITR and the 3′ end of the right ITR) are located in proximity to one another while the heads of each ITR are separated and face outward.

The “encapsidation signal of adenovirus” or “adenovirus packaging sequence” refers to the ψ sequence which comprises five (AI-AV) packaging signals and is required for encapsidation of the mature linear genome; the packaging signals are located from about 194 to 358 bp in the Ad genome (about 0.5-1.0 mu).

The viral backbone shuttle vector may contain multiple restriction endonuclease sites for the insertion of a foreign DNA sequence of interest. The foreign DNA sequence of interest typically comprises cDNA or genomic fragments that are of interest to transfer into mammalian cells. Foreign DNA sequence of interest may include any naturally occurring or synthetic DNA sequence. The foreign DNA may be identical in sequence to naturally-occurring DNA or may be mutated relative to the naturally occurring sequence. The foreign DNA need not be characterized as to sequence or function.

The size of foreign DNA that may be included in the shuttle vector will depend upon the size of the rest of the vector. If necessary, the HPRT introns may be removed to adapt large size foreign DNA fragment. The total size of foreign DNA may vary from 1 kb to 35 kb.

The foreign DNA may encode protein, or contain regulatory sites, including but not limited to, transcription factor binding sites, promoters, enhancers, silencers, ribosome binding sequences, recombination sites, origins of replication, sequences which regulate RNA stability and polyadenylation signals. The promoters used may vary in their nature, origin and properties. The choice of promoter depends in fact on the desired use and on the gene of interest, in particular. Thus, the promoter may be constitutive or regulated, strong or weak, ubiquitous or tissue/cell-specific, or even specific of physiological or pathophysiological states (activity dependent on the state of cell differentiation or the step in the cell cycle). The promoter may be of eukaryotic, prokaryotic, viral, animal, plant, artificial or human, etc., origin. Specific examples of promoters are the promoters of the genes PGK, TK, GH, α-EF1, APO, CMV, etc. or artificial promoters, such as those for p53, E2F or cAMP.

2. Adeno-associated viruses (AAV) vectors: AAV is a naturally occurring defective virus that requires another virus, such as an adenovirus or a herpes virus, as a helper virus for efficient replication and a productive life cycle (Muzyczka et al., Curr. Topics in Micro. and Immunol. 158:97-129, [1992]). AAV vector is the only viral vector system that is based on a non-pathogenic and replication defective virus. It is also one of the few viruses that may integrate its DNA into non-dividing cells, and exhibits a high frequency of stable integration (Flotte et al., Am. J. Respir. Cell. Mol. Biol. 7:349-356, [1992]; Samulski et al., J. Virol. 63:3822-3828, [1989]). Vectors containing as little as 300 base pairs of AAV DNA can be packages.

AAV vectors have been successfully used to establish efficient and long-term gene expression in vivo in a variety of tissues without significant immune response or toxicity (Xiao et al., J. Virol. 70:8098-108, [1996]; Kessler et al., Proc Natl Acad Sci USA 93, 14082-7, [1996]; Xiao et al., J Virol 72:10222-6, [1989]). Unlike other viral vectors, AAV readily bypasses extracellular barriers due to its small viral particle size (20 nM) that facilitates efficient transduction of muscle myofibers of various maturity (Pruchnic et al., Hum Gene Ther, 11:521-36, [2000]). However, a major obstacle for AAV vectors is the limited packaging size that only allows for genes smaller than 4.7 kb (Song et al., Proc Natl Acad Sci USA 95:14384-8, [1998]; Kay et al., Nat Genet 24:257-261, [2000]), therefore precludes such large gene as dystrophin with a CANA of 14 kb.

3. Herpes simplex virus (HSV) vectors: The main feature of an HSV vector is that it has very large packaging capacity, is usually replication defective, and does not integrate into the host genome. HSV infects cells of the nervous system (Fink et al., Annu Rev Neurosci 19:265-287, [1996]). The virus contains more than 80 genes, one of which (IE3) can be replaced to create the vector. The generation of HSV vectors with deletions in many of the immediate early gene products has resulted in vectors with reduced toxicity and antigenicity, as well as prolonged expression in vivo. However, these modifications also result in a lower virus yield. Construction of HSV vectors is described in U.S. Pat. No. 5,661,033.

4. Retrovirus vectors: Defective retroviruses are well characterized for use in gene transfer for gene therapy purposes (Miller Blood 76:271-278, [1990]). The members of the family Retroviridae are characterized by the presence of reverse transcriptase in their virions. There are several genera included within this family, including Cistemavirus A, Oncovirus A, Oncovirus B, Oncovirus C, Oncovirus D, Lentivirus, and Spumavirus.

A recombinant retrovirus can be constructed having a nucleic acid encoding a gene product of interest inserted into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in “Current Protocols in Molecular Biology, Ausubel, et al., (eds.) Greene Publishing Associates, (1989), Sections 9.10-9.14” and other standard laboratory manuals. Examples of suitable retroviruses include pLJ, PZIP, pWE and pEM which are well known to those skilled in the art. Examples of suitable packaging virus cell lines include psi.Crip, psi.Cre, psi.2 and psi.Am. Retroviruses have been used to introduce a variety of genes into many different cell types, including epithelial cells, endothelial cells, lymphocytes, myoblasts, hepatocytes, hematopoietic stem cells, in vitro, and/or in vivo (U.S. Pat. No. 4,868,116; U.S. Pat. No. 5,449,614 and U.S. Pat. No. 6,207,455). Retroviral vectors require target cell division in order to be integrated into the host genome to stable introduce nucleic acid into the cell. Thus, it may be necessary to stimulate replication of the target cell. Successful transduction of hematopoietic stem or progenitor cells with retroviral vectors in an ex vivo setting have been reported. However, Recombinant retroviral vectors can only accommodate about 8 kb to 10 kb of foreign DNA, and this packaging capacity limits its use.

5. Lentivirus vectors: Lentivirus also belong to the retrovirus family, but they can infect both dividing and non-dividing cells. The best-known lentivirus is the human immunodeficiency virus (HIV), which has been disabled and developed as a vector for in vivo gene delivery. Like the simple retroviruses, HIV has the three gag, pol and env genes, but it also carries genes for six accessory proteins tenned tat, rev, vpr, vpu, nef and vif. Using the retrovirus vectors as a model, lentivirus vectors have been made, with the transgene enclosed between the LTRs and a packaging sequence (Naldni et al., Science 272:263-267, [1996]). Some of the accessory proteins can be eliminated without affecting production of the vector or efficiency of transfection.

When lentiviral vectors are injected into rodent brain, liver, muscle, or pancreatic islet cells, they give sustained expression for over six months. Little is known about the possible immune problems associated with lentiviral vectors. Furthermore, there seems to be no potent antibody response. A major concern about lentiviral vector is its safety in human applications. However, recent development in producing the third generation lentiviral vectors with more deletion in viral genes and improved safety may allow for the general application of lentiviral vectors to in vivo gene therapy.

Other viral vector systems that may have application in the subject invention have been derived from vaccinia virus (Chen et al., J. Immunother 24:46-57, [2001]), and several RNA viruses. The plus-strand RNA viridae, such as polio (Bledsoe et al., Nat Biotechnol. 18:964-9, [2000]), hepatitis A (Romano G. Stem Cells; 18:19-39, [2000]), and sindbis virus (Wahlfors et al., Gene Ther 7:472-80, [2000]) are being developed for high-level gene expression, following either viral infection or delivery of nucleic acids using a non-viral system. These viruses express a replicase protein that can specifically replicate the viral RNA. By inserting a transgene in place of the viral capsid gene(s), it is possible to generate a chimeric RNA that replicates autonomously in the cell and expresses a high level of protein from the plus-coding strand of RNA. These viral vectors are well suited for immunization strategies in which high, transient gene expression is needed to induce an immune response to the transduced cells.

In addition to the viral gene transfer vectors, powerful non-viral gene transfer vectors have also become available for clinical application in the past several years (Ropert et al., Braz J Med Biol Res. 32:163-9, [1999]; Lee et al., Crit Rev Ther Drug Carrier Syst 14:173-206, [1997]). These vectors rely on normal mechanisms used by mammalian cells for the uptake and intracellular transport of macromolecules to deliver genetic materials into cells. Commonly used non-vector include cationic and other liposomes.

Liposomes are formulated based on the requirement of the delivery system in a particular application. The characteristics of liposomes, such as size and composition, can be modified during the preparation of the liposomes.

Typically, liposomes are prepared by dissolving one or more lipids in an organic solvent. The solvent is evaporated under controlled conditions resulting in a uniform, thin lipid layer of lipid mix in the evaporating flask. Phosphate buffered saline or water is added to the dried lipid mix layer in the evaporating flask and is sonicated briefly to form a liposome suspension. The preparation is dehydrated, rehydrated and stored at 4° C.

The lipids may be natural, synthetic or semisynthetic (i.e., modified natural). Lipids useful in the invention include, and are not limited to, fatty acids, lysolipids, oils (including safflower, soybean and peanut oil), phosphatidylcholine with both saturated and unsaturated lipids. The lipids also include cationic lipids and synthetic cationic lipids. The lipids may also include derivatized lipids, including common natural lipids derivatized to contain one or more basic functional groups. Additionally lipid moieties capable of polymerization may be used as coatings for the liposomes. Examples of these include, but are not limited to, alkenyl and alkynyl moieties, such as oleyl and linoleyl groups, diacetylene, acryloyl and methacryloyli groups with or without polar groups to enhance water solubility.

Other non-viral vectors include DNA-viral conjugates, RNA/DNA oligonucleotides and naked DNA molecules. Physical procedures, such as hydrodynamics-based and electroporation-based procedures, have been used to improve gene transfer efficiency of some non-viral vectors (Zhang et al., Gene Ther 7:1344-9, [2000]; Yamashita et al., Cancer Res. 61:1005-12, [2001]). Recently, it was also reported that intraperitoneal injection of a β-galactosidase fused to the protein transduction domain from the human immunodeficiency virus TAT protein resulted in delivery of the fusion protein to all tissues in mice (Schwarze et al., Science, 3:1569-1572, [1999]).

In vitro expression of thrombomodulin or a thrombomodulin variant may also be achieved with traditional transfection methods such as calcium phosphate precipitation, DEAE-dextron transfection, and electroporation.

Another aspect of the invention pertains to the expression of thrombomodulin or a thrombomodulin variant using a regulatable expression system. Systems to regulate expression of therapeutic genes have been developed and incorporated into the current viral and non-viral gene delivery vectors. These systems are briefly described below:

Tet-on/off system. The Tet-system is based on two regulatory elements derived from the tetracycline-resistance operon of the E. coli Tn 10 transposon: the tet repressor protein (TetR) and the Tet operator DNA sequence (tetO) to which TetR binds. The system consists of two components, a “regulator” and a “reporter” plasmid. The “regulator” plasmid encodes a hybrid protein containing a mutated Tet repression (tetr) fused to the VP 16 activation domain of herpes simplex virus. The “reporter” plasmid contains a tet-responsive element (TRE), which controls the “reporter” gene of choice. The tetr-VP16 fusion protein can only bind to the TRE, therefore activate the transcription of the “reporter” gene, in the presence of tetracycline. The system has been incorporated into a number of viral vectors including retrovirus, adenovirus and AAV (Gossen and Bujard, Proc. Natl. Acad. Sci. USA 89:5547-5551, [1992]; Gossen et al., Science 268:1766-1769, [1995]; Kistner et al., Proc. Natl. Acad. Sci. USA. 93:10933-10938, [1996]).

Ecdysone system. The Ecdysone system is based on the molting induction system found in Drosophila, but modified for inducible expression in mammalian cells. The system uses an analog of the drosophila steroid hormone ecdysone, muristerone A, to activate expression of the gene of interest via a heterodimeric nuclear receptor. Expression levels have been reported to exceed 200-fold over basal levels with no effect on mammalian cell physiology (No et al., Proc. Natl. Acad. Sci. USA 93:3346-3351, [1996]).

Progesterone-system. The progesterone receptor is normally stimulated to bind to a specific DNA sequence and to activate transcription through an interaction with its hormone ligand. Conversely, the progesterone antagonist mifepristone (RU486) is able to block hormone-induced nuclear transport and subsequent DNA binding. A mutant form of the progesterone receptor that can be stimulated to bind through an interaction with RU486 has been generated. To generate a specific, regulatable transcription factor, the RU486-binding domain of the progesterone receptor has been fused to the DNA-binding domain of the yeast transcription factor GAL4 and the transactivation domain of the HSV protein VP16. The chimeric factor is inactive in the absence of RU486. The addition of hormone, however, induces a conformational change in the chimeric protein, and this change allows binding to a GAL4-binding site and the activation of transcription from promoters containing the GAL4-binding site (Wang et al., Proc. Natl. Acad. Sci. USA 93:8180-8184, [1994]; Wang et al., Nat. Biotech 15:239-243, [1997]).

Rapamycin-system. Immunosuppressive agents, such as FK506 and rapamycin, act by binding to specific cellular proteins and facilitating their dimerization. For example, the binding of rapamycin to FK506-binding protein (FKBP) results in its heterodimerization with another rapamycin binding protein FRAP, which can be reversed by removal of the drug. The ability to bring two proteins together by addition of a drug potentiates the regulation of a number of biological processes, including transcription. A chimeric DNA-binding domain has been fused to the FKBP, which enables binding of the fusion protein to a specific DNA-binding sequence. A transcriptional activation domain also has been used to FRAP. When these two fusion proteins are co-expressed in the same cell, a fully functional transcription factor can be formed by heterodimerization mediated by addition of rapamycin. The dimerized chimeric transcription factor can then bind to a synthetic promoter sequence containing copies of the synthetic DNA-binding sequence. This system has been successfully integrated into adenoidal and AAV vectors. Long-term regulatable gene expression has been achieved in both mice and baboons (Magari et al., J. Clin. Invest. 100: 2865-2872, [1997]; Ye et al., Science 283:88-91, [1999]).

Another aspect of the invention pertains to isolated polynucleotide molecules., which may be used to reduce or to eliminate a thrombomodulin or a thrombomodulin variant. One method of reducing or eliminating TM gene expression is to introduce an antisense TM construct into a mammal.

An “antisense” polynucleotide comprises a nucleotide sequence, which is complementary to a “sense” polynucleotide encoding a protein, e.g., complementary to the coding strand of a double-stranded cDNA molecule or complementary to an mRNA sequence. Accordingly, an antisense polynucleotide can hydrogen bond to a sense polynucleotide. The antisense polynucleotide can be complementary to an entire coding strand of a gene of the invention or to only a portion thereof. In one embodiment, an antisense polynucleotide molecule is antisense to a “coding region” of the coding strand of a nucleotide sequence of the invention. The term “coding region” includes the region of the nucleotide sequence comprising codons, which are translated into amino acid. In another embodiment, the antisense polynucleotide molecule is antisense to a “noncoding region” of the coding strand of a nucleotide sequence of the invention.

Antisense polynucleotides of the present invention can be designed according to the rules of Watson and Crick base pairing. The antisense polynucleotide molecule can be complementary to the entire coding region of an mRNA corresponding to a gene of the invention, but more preferably is an oligonucleotide, which is antisense to only a portion of the coding or noncoding region. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions using procedures known in the art. For example, an antisense polynucleotide (e.g., an antisense oligonucleotide) can be chemically synthesized using naturally occurring nucleotides or variously modified nucleotides designed to increase the biological stability of the molecules or to increase the physical stability of the duplex formed between the antisense and sense polynucleotides, e.g., phosphorothioate derivatives and acridine substituted nucleotides can be used. Examples of modified nucleotides which can be used to generate the antisense polynucleotide include 5-fluorouracil, 5-bromouracil, 5-chlorouracil, 5-iodouracil, hypoxanthine, xantine, 4-acetylcytosine, 5-(carboxyhydroxyhnethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl2-thiouracil, beta-D-mannosylqueosine, 5′-methoxycarboxymethyluracil, 5-methoxyuracil, 2-methylthio-N-6-isopentenyladen4exine, unacil-5-oxyacetic acid (v), wybutoxosine, pseudouracil, queosine, 2-thiocytosine, 5-methyl-2-thio-aracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense polynucleotide can be produced biologically using an expression Vector into which a polynucleotide has been subcloned in an antisense orientation (i.e., RNA transcribed from the inserted polynucleotide will be of an antisense orientation to a target polynucleotide of interest, described further in the following subsection).

The antisense polynucleotide molecules of the present invention are typically administered to a mammal or generated in situ such that they hybridize with or bind to cellular mRNA and/or genomic DNA encoding a thrombomodulin or a thrombomodulin variant to thereby inhibit expression of the protein, e.g., by inhibiting transcription and/or translation. The hybridization can be by conventional nucleotide complementarity to form a stable duplex, or, for example, in the cases of an antisense polynucleotide molecule which binds to DNA duplexes, through specific interactions in the major groove of the double helix. An example of a route of administration of antisense polynucleotide molecules of the invention is direct injection at a tissue site (e.g., intestine or blood). Alternatively, antisense polynucleotide molecules can be modified to target selected cells and then administered systemically. The antisense polynucleotide molecules can also be delivered to cells using the vectors described herein. To achieve sufficient intracellular concentrations of the antisense molecules, vector constructs it which the antisense polynucleotide molecule is placed under the control of a strong promoter are preferred.

Expression of the TM gene can also be inhibited using RNA interference (“RNAi”). RNAi is a phenomenon of the introduction of double-stranded RNA (dsRNA) into certain organisms and cell types causes degradation of the homologous mRNA.

RNAi was first discovered in the nematode Caenorhabditis elegans, and it has since been found to operate in a wide range of organisms. In recent years, RNAi has becomes an endogenous, efficient, and potent gene-specific silencing technique that uses double-stranded RNAs (dsRNA) to mark a particular transcript for degradation in vivo. RNAᵢ technology is disclosed, for example, in U.S. Pat. No. 5,919,619 and PCT Publication Nos. WO99/14346 and WO01/29058.

Briefly, dsRNAs 21-25 nucleotides long, called short interfering RNAs (siRNA), are introduced into the cell. SiRNAs may also be produced endogenously by degradation of long dsRNA molecules by an RNAse III-related nuclease called Dicer. Once formed, the siRNAs assemble with protein components into an RNA-induced silencing complex (RISC). An ATP-generated unwinding of the siRNA activates the RISC, which in turn targets the homologous mRNA transcript by Watson-Crick base-pairing and cleaves the mRNA. This sequence specific degradation of mRNA results in gene silencing.

Gene transfer vectors can be delivered to a mammal by, for example, intravenous administration, intraportal administration, intrabiliary administration, intra-arterial administration, direct injection into the liver parenchyma (see U.S. Pat. No. 6,328,958), by intramusclular injection (see U.S. Pat. No. 6,335,011), by inhalation (see U.S. Pat. No. 6,344,194), by perfusion (U.S. Pat. No. 6,342,214) or by stereotactic injection (see e.g., Chen et al., Proc. Natl. Acad. Sci. USA 91:3054-3057, [1994]). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The invention is further directed to pharmaceutical compositions comprising a gene transfer vector described hereinabove and a pharmaceutically acceptable carrier.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, Ind the like, compatible with pharmaceutical administration. The use of such media and agents for pharmaceutically active substances is well-known in the art. [See e.g., A. H. Kibbe Handbook of Pharmaceutical Excipients, 3rd ed. Pharmaceutical Press London, UK (2000)]. Except insofar as any conventional media or agent is incompatible with the active compound, use thereof in the compositions is contemplated. Supplementary agents can also be incorporated into the compositions.

The invention includes methods for preparing pharmaceutical compositions for modulating the expression or activity of thrombomodulin. Such methods comprise formulating a pharmaceutically acceptable carrier with a gene transfer vector capable of modulating expression or activity of thrombomodulin. Such compositions can further include additional active agents. Thus, the invention further includes methods for preparing a pharmaceutical composition by formulating a pharmaceutically acceptable carrier with a gene transfer vector capable of modulating expression or activity of thrombomodulin and one or more additional bioactive agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylene-diaminetetracetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active ingredient (e.g., a viral or non viral vector) in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active ingredient can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrans appropriate to the barrier to be permeated are used in the formulation. Such penetrans are generally known in the art, and include, for example, for transmucosal administration, detergents, bile salts, and fusidic acid derivatives. Transmucosal administration can be accomplished through the use of nasal sprays or suppositories. For transdermal administration, the bioactive ingredient are formulated into ointments, salves, gels, or creams as generally known in the art.

The composition can also be prepared in the form of suppositories (e.g., with conventional suppository bases such as cocoa butter and other glycerides) or retention enemas for rectal delivery.

It is especially advantageous to formulate oral or parenteral compositions in dosage unit form for ease of administration and uniformity of dosage. Dosage unit form as used herein includes physically discrete units suited as unitary dosages for a mammal to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active ingredient and the particular therapeutic effect to be achieved, and the limitations inherent in the art of compounding such an active ingredient for the treatment of individuals.

Toxicity and therapeutic efficacy of such ingredient can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration arrange that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. The pharmaceutical compositions can be included in a container, pack, or dispenser together with instructions for administration.

In one embodiment, the in vivo expression of thrombomodulin or a thrombomodulin variant is used for the treatment of atherosclerotic cardiovascular disease (CVD). Though venous grafts can be used for bypass surgeries, the veins eventually, become occluded by thrombosis resulting the recurrence of the diseases. TM gene delivery can be used in coronary artery bypass grafting, percutaneous transluminal coronary angioplasty, peripheral artery angioplasty or thrombectomy, intravascular stenting and vascular graft prostheses to block thrombosis. TM gene delivery can be also used for the reduction of non-intima formation, for the prevention of atherosclerosis; for the prevention of myocardial infarction and for the inhibition of fibrinolysis in hemophilic plasma. TM gene transfer at the site of thrombus; formation is potent approach to reverse these vascular diseases.

In another embodiment, the in vivo expression of thrombomodulin or a thrombomodulin variant is used for the treatment of pulmonary hypertension. Reduction of TM levels cause altered homeostasis in pulmonary hypertension. Therefore, in vivo TM expression can be used to correct this disease state.

In another embodiment, the in vivo expression of thrombomodulin, or a thrombomodulin variant is used for the treatment of end-stage renal failure disease. (ESRD). ESRD patients often exhibit decreased antithrombotic activity due to low TM levels. In such patients, enhanced in vivo TM gene expression can be potentially very useful.

In yet another embodiment, the in vivo expression of thrombomodulin or a thrombomodulin variant is used for the treatment of acute inflammatory diseases such as Sepsis. In sepsis, liver participates in host defense and tissue repair through hepatic cross talk that controls coagulation and inflammatory processes. In the absence of this control, it can lead to bacterial spill over, enhanced procoagulant and inflammatory process. This can result in multiple organ failure and death. TM can be used to bock septic shock induced by variety of bacterial and other infections.

In yet another embodiment, the in vivo expression of thrombomodulin or a thrombomodulin variant is used for the treatment of Alzheimer's disease (AD).

Studies have shown that vascular risk factors are also involved in early Alzheimer's disease. Thus TM gene transfer can be also useful in reversing, inhibiting AD progression.

In another embodiment, the in vivo expression of thrombomodulin inhibitory polynucleotide is used for the treatment of the diseases/conditions relating to an overexpression of thrombomodulin.

The present invention is further illustrated by the following examples which should not be construed as limiting. The contents of all references, patents and published patent applications cited throughout this application, as well as the Figures and Tables are incorporated herein by reference.

### EXAMPLE 1

### Construction of Gutless Viral Backbone Shuttle Vector

An embodiment of a gutless viral backbone shuttle vector pShuttle is shown in FIG. 1. The shuttle vector pShuttle has a total length of 13602 bp (SEQ ID NO: 1). Sequence portion containing R-ITR, PBR322 ori, Kan, L-ITR, and encapsidation signal was obtained from the pAdEasy system from Stratagene. At bp 3667 of the original pShuttle sequence, there is a BamHI site just beyond the R-ITR. PCR primers were designed to include the BamHI site and then was to create an EcoRI site at the end of the R-ITR. The R-ITR was PCR replicated and then digested with BamHI and EcoRI to create sticky ends. The viral backbone was then cut with both BamHI and EcoRI. The BamHI cut the backbone at bp 3667 and there was also an EcoRI site inside the MCS at bp 377. The backbone portion of the plasmid was then gel purified and the PCR replicated R-ITR was recloned into position. This essentially puts the L-ITR, encapsidation signal, MCS, and R-ITR all in close proximity to each other.

Insertion of the HPRT introns was a two step cloning process. First, the viral backbone was digested with EcoRI and XbaI, both enzyme sites are in the MCS. The HPRT source was also digested with EcoRI and XbaI yielding a 7477 bp fragment that was cloned into the EcoRI/XbaI digested viral backbone. Then the HPRT source was digested with only XbaI yielding a 2715 bp fragment. One of the XbaI sites in this cut is the same XbaI site that was cut from the EcoRI/XbaI double digest in step 1. The viral backbone was cut with only XbaI and the 2715 bp fragment was inserted.

Overall, from the HPRT source, the HPRT stuffer sequence is inserted into the viral backbone in reverse orientation, hence intron 5, then 4, then 3. The 2715 bp fragment was inserted and checked to follow the original source sequence.

### EXAMPLE 2

### Construction and Preparation of Gutless Viral Shuttle Vector

(a) Construction and Preparation of Gutless Viral Shuttle Vector Carrying Human Thrombomodulin (hTM) Gene

The insertion of hTM gene into the gutless adenovirus backbone first required the creation of a CMV-hTM expression cassette.

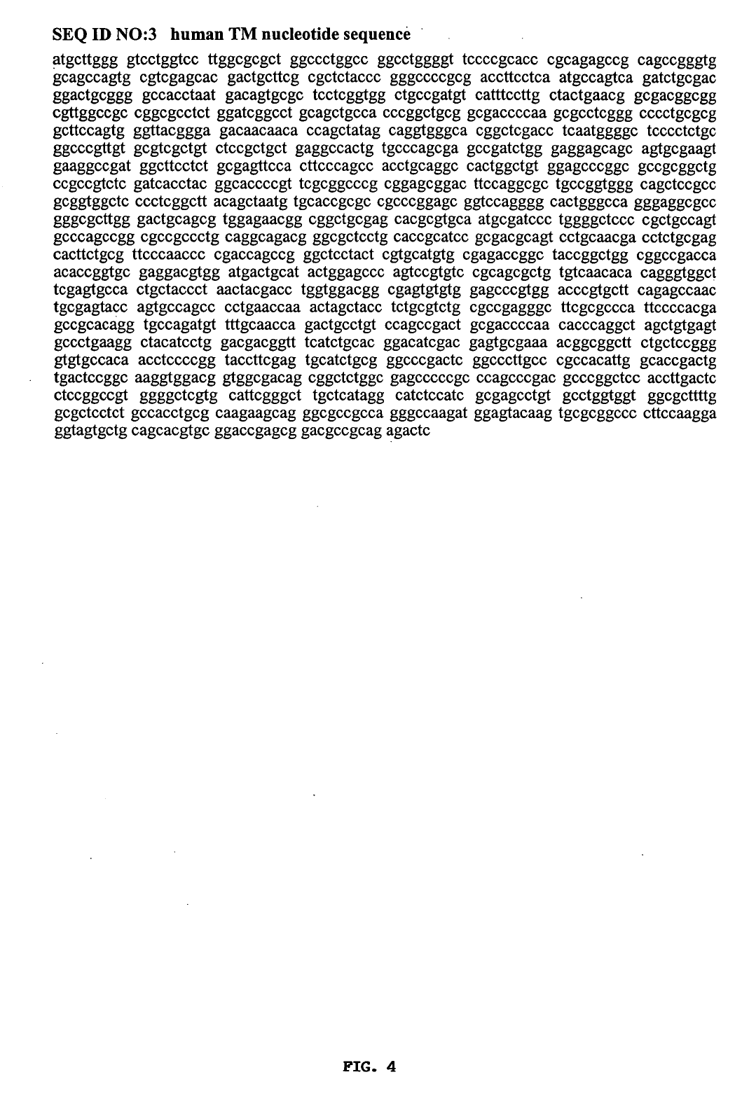

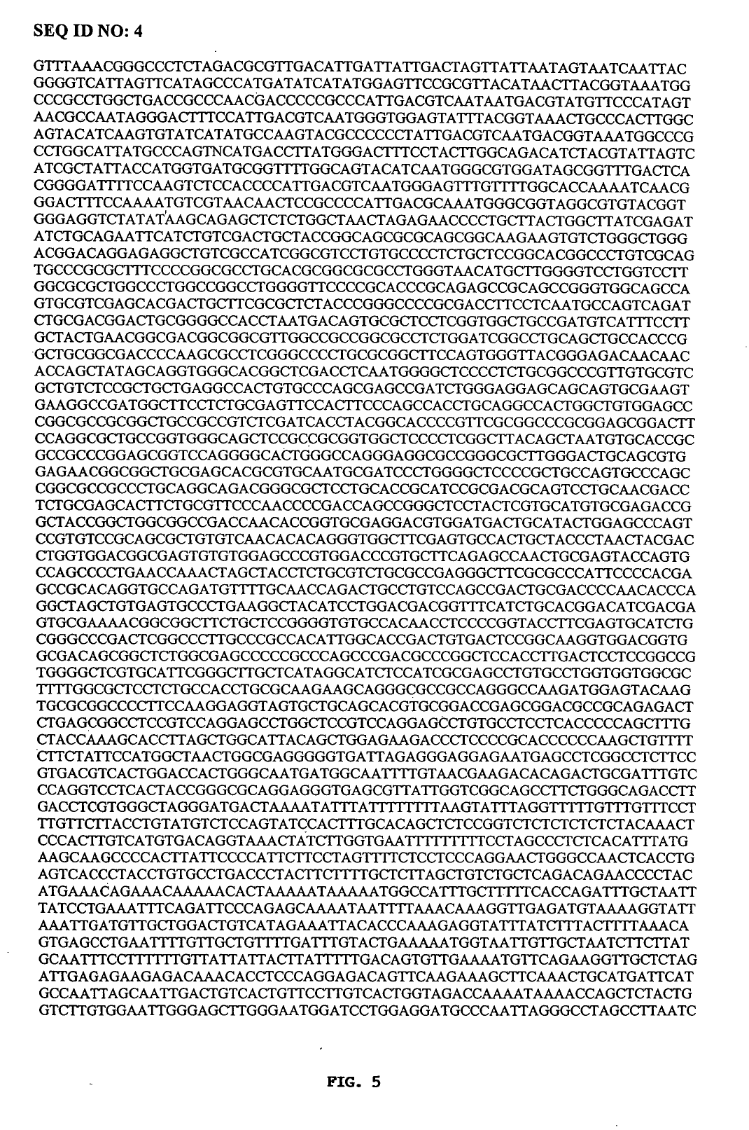

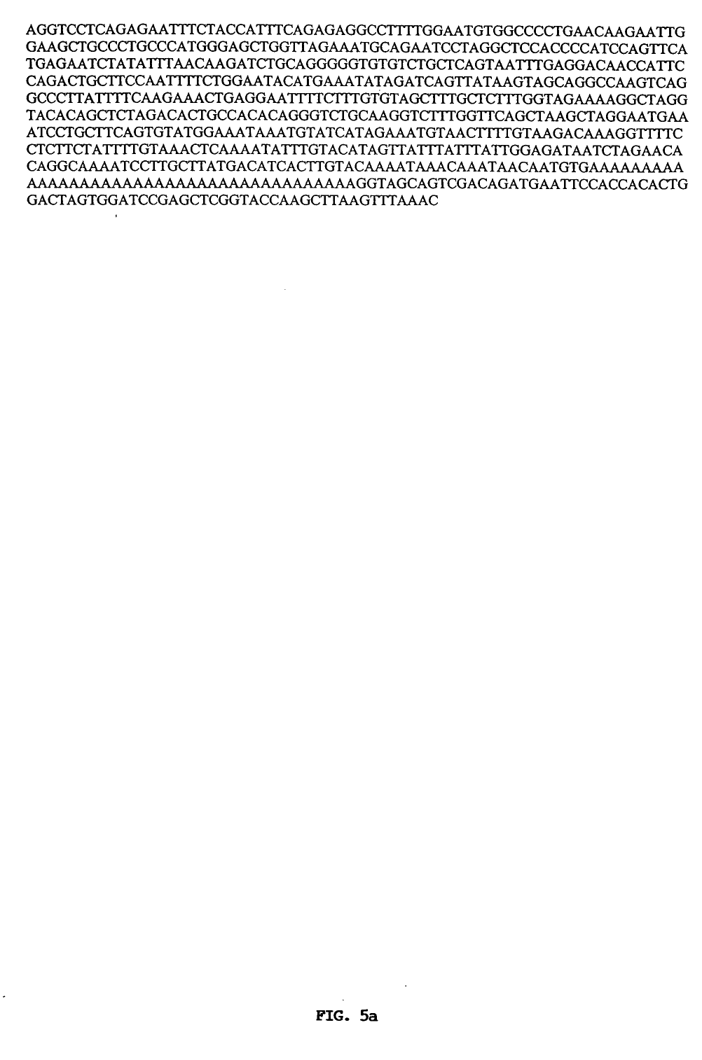

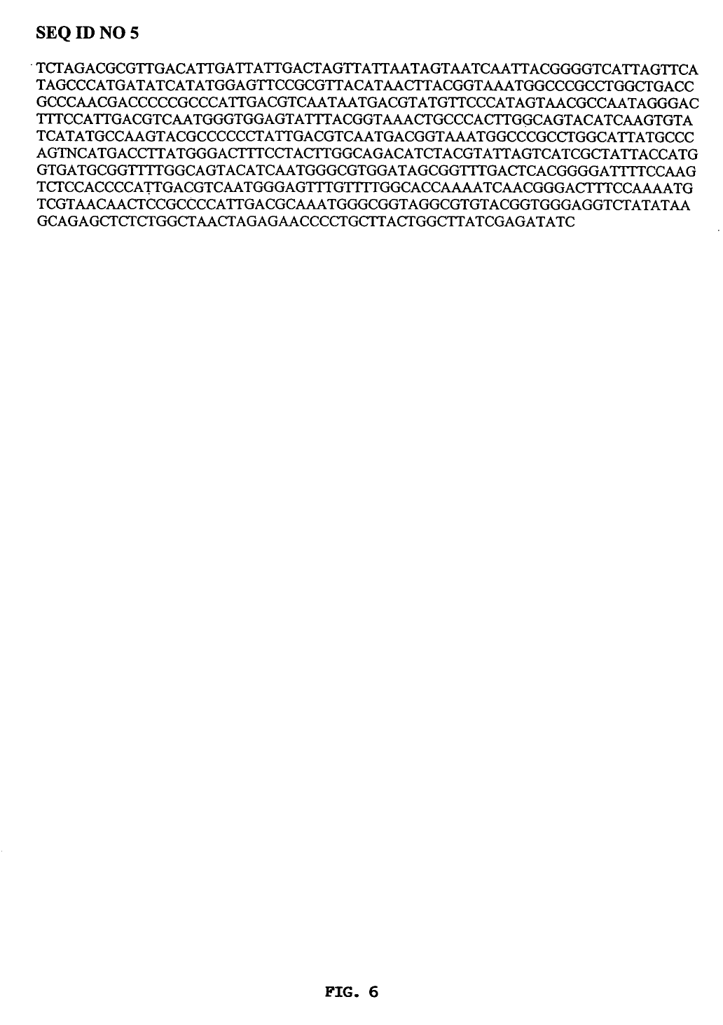

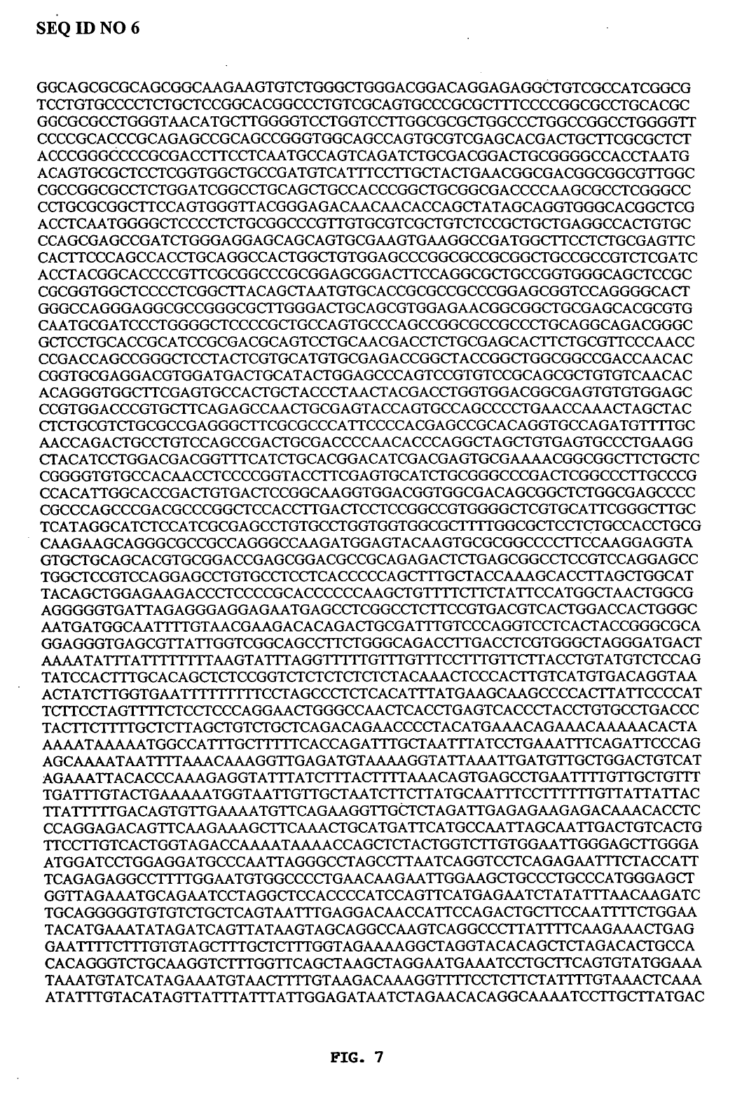

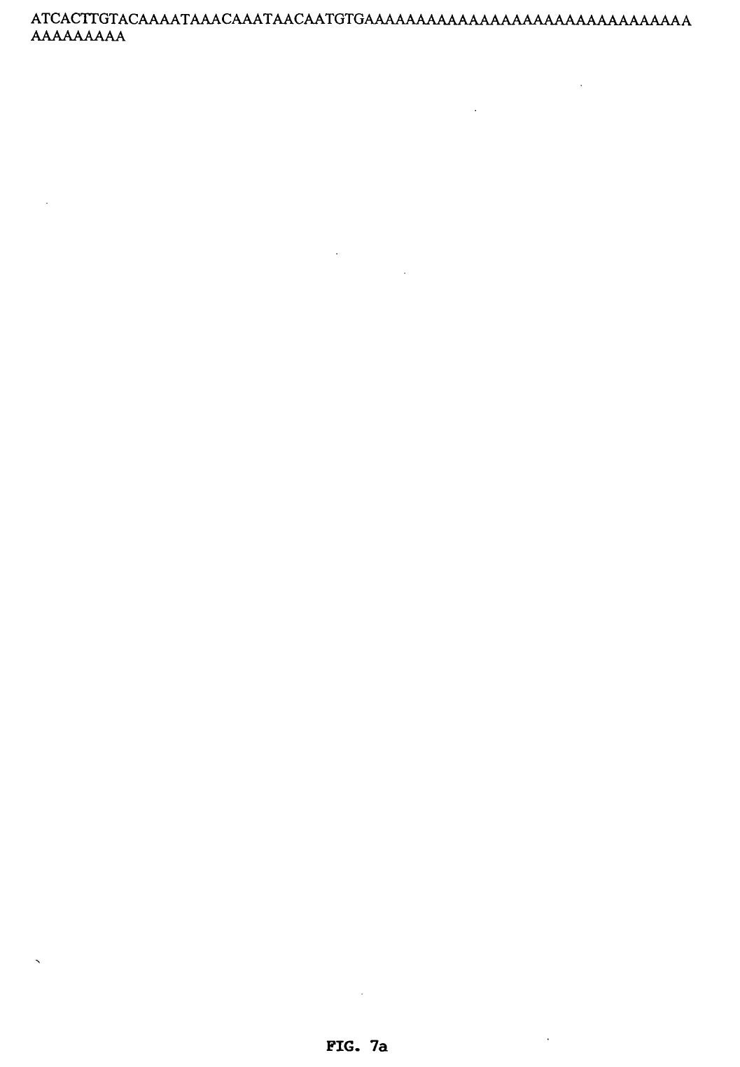

The intermediate vector used was pcDNA3.1/Zeo(+) (Invitrogen). A CMV promoter is available commercially and a CMV promoter was cloned into the multiple cloning site (MCS) at the Xbal/EcoRV restriction enzyme site locations. The CMV from ps5 was removed using XbaI/EcoRV. pcDNA3.1/Zeo(+) was cleaved inside the MCS using both XbaI and EcoRV as well. The CMV promoter was then ligated. Due to the location of the enzyme sites in the MCS, the CMV promoter (FIG. 6, SEQ ID NO:5) was inserted in a backwards orientation relative to the pcDNA3.1/Zeo(+) plasmid. The TM cDNA (FIG. 7, SEQ ID NO:6) was obtained from Dr. Sadler (Dittman et al., Biochemistry, 26(14):4350-4357 [1987]) which the sequence was also submitted to ATCC and to GenBank. The TM gene was removed from the plasmid using EcoRI and inserted into pcDNA3.1/Zeo(+), also in the reverse orientation to pcDNA3.1/Zeo(+) downstream of the inserted CMV promoter. To remove the cassette, PmeI enzyme was used to cut both ends of the MCS. The gutless adenovirus backbone was linearized using SmaI which is at bp 381 of the backbone. The two were ligated together in the forwards orientation with respect to the gutless virus backbone. Sequence of the expression cassette (from PmeI site to PmeI site, SEQ ID NO:4) is shown in FIG. 5.

(b). Construction and Preparation of Gutless Viral Shuttle Vector Carrying LacZ Gene

The insertion of LacZ also required creation of an intermediate vector to create the expression cassette. pcDNA3.1/Zeo(+) was again used. First, a portion of the vector from the end of the MCS, restriction enzyme site Apal, to the beginning of the SV40 poly A, restriction site Nael, was removed and the vector relegated to itself. Then the LacZ gene was inserted into the vector MCS using NotI/XbaI. The expression cassette, containing CMV promoter, LacZ gene, and SV40 poly A, was removed using Nrul/Sall retraction enzymes and blunt-end cloned into the gutless adenovirus at the Smal restriction enzyme site.

### EXAMPLE 3

### Preparation of Gutless Adenovirus

The helper virus was an E1/E3 deleted adenovirus in which a special flp recognition sequence site (FRS) flanks the encapsidation signal. Helper adenovirus need to be grown in 293 cells.

293 cell line has long ago been engineered to express E1 and E3 genes of adenovirus. These two genes are necessary for viral reproduction. The flp gene is similar in function to Cre-Lox. The flp gene will recognize the FRS, cleave at that location, and then relegate the DNA. It's basic function is to promote recombination between different pieces of DNA with the FRS, but in this case, it will cleave out the encapsidation signal thereby not allowing helper-viral DNA to be packaged. [Beauchamp et al., Molecular Therapy, 3(5):809-815 (2001); Umana et al., Nature Biotechnology, 19:582-585 (2001)].

293-flp cells were transfected with the backbone DNA using Lipofectamine. While performing the transfection, helper virus were used to infect the 293-flp cells. The helper virus inserted its own DNA into the 293-flp cells. The flp protein expressed in the cells cleaved the encapsidation signal thereby not allowing the helper virus DNA to package. Consequently, the gutless adenovirus backbone DNA was packaged into the adenoviral proteins expressed from the helper virus DNA and formed gutless adenovirus (gutless Ad hTM or gutless Ad lacZ). The gutless viruses contain the hTM or lacZ expression cassette but could nor replicate in normal cells due to the E1/E3 deletions. The virus were produced by the following procedure:

(a) Virus Reproduction

Seed 293 cells in 15 cm dishes and grow in 10% FBS until approximately 70% confluent. Viral media was made as follows: 2 ml of FBS-free IMEM containing antibiotic, antimycotic; adjust pfu per cell of purified virus until reached the final concentration of media as 1 μl virus in 2 ml IMEM (viral Conc. 1×10¹⁰ pfu/ml)/each 15 cm dish. For Example: 30 Dishes=60 ml IMEM+30 μl virus Old media was Aspirated from dishes, and 2 ml viral media was added per dish. Dishes were rocked at 37° C. for 1.5-2 hours, and 18 mL 10% media was added per dish and incubated according to time course.

Cells were harvested by pipeting and collocating in 50 ml tubes at 4° C., and cells were centrifuge at 4° C., 2000 rpm for 5 min. Save 10 ml of supernatant from one of the tubes into a separate tube. The supernatant was removed from all of the tubes. Take 5 mL supernatant from the saved tube and resuspend all the pellets to one tube. All of the tubes were re-wash with the remaining 5 mL of supernatant to collect any leftover sample, and the pellet was store at −80° C.

(b) Virus Collection

Sample tube(s) were frozen/thawed 5 times to lyse the cells, and the virus were released using dry ice and incubated at 37° C. water bath for 15 minutes until each to obtain crude viral lysate (CVL). The CVL was collected in two 2059 Falcon Tubes and centrifuged using Sorvall HS4 at 7000 rpm, 4° C. for 5 minutes and the supernatant was recovered.

To purify the virus, ultra-clear SW41 (Beckman) tubes were prepared by soaking in Ultra Pure Water, then 70% ETOH. Cotton swabs (one swab for each tube) was used to completely dry out the tube, and two tubes were used per sample.

Preparation of the first gradient: 2.5 ml CsCl—Density 1.25, and 2.5 ml CsCl—Density 1.40. Place the 1.25 density CsCl into the Beckman tubes first. Underlay slowly the high density, 1.40 CsCl using a sterile pasteur pipette, and overlay an equal amount (in ml) of CVL, about 4.25 ml/tube. Samples were centrifuged in a SW41 rotor with speed: 35,000 rpm at 20° C. for 1 hour and with acceleration: 1 and deceleration: 4. The lower opalescent band was collected using 1 or 3 ml syringe with green cap needles.

Preparation of second gradient: CsCl was prepared to density 1.33. Two fresh ultra-clear tubes were placed 8 ml of CsCl and overlay the band just recovered after the first spin. (To equilibrate the tubes, measure before the volume of the recovered band and divide equally in the 2 tubes). Samples were centrifuged at the conditions above for 18 hours. The opalescent band was recovered and collected in a sterile eppendorf tube. (From this moment, keep the tube always on ice). Samples were dialyze with dialysis buffer: (1) 10× Dialysis Buffer: 100 mM Tris-pH 7.4, 10 mM MgCl₂; (2) 1× Dialysis Buffer (2 Liters): 400 ml Glycerol, 200 ml 10× Dialysis Buffer 140 ml, and Ultra Pure Water. The dialyzed samples were immediately stored at −70° C.

Alternative, the virus can be purified using column chromatography. Such method has been described, for example, by Sakhuja et al and Green et al [Sakhuja et al., Human Gene Therapy, 14:243-254 (2003); Green et al., Human Gene Therapy, 13:1921-1934, (2002)]. Purification kit for adenovirus using column chromatography is also commercially available, e.g., the ViraKit from Virapur, LLC (San Diego, Calif.). Briefly, the infected cell will be harvested and lysed by several freeze-and-thaw cycles. The cell debris will be precipitated by centrifugation. The supernatant will be collected and clarified by passing through a 0.45 micron clarification filter. The clarified supernatant will be treated with DNase and then applied to a purification filter by centrifugation. After two or more washes, the virus will be eluted from the purification filter. The protein concentration of the eluant will be determined using a BioRad protein estimation kit and the following formula will be used to convert protein concentration to titer: [12.956+224.15 (μg/ml)]×10⁸.

### EXAMPLE 4

### Expression of hTM In Vitro Using Gutless Ad hTM

When enough gutless Ad hTM has been produced, experiments will be performed to demonstrate the viable expression of hTM in vitro with gutless Ad hTM in cultured human cells such as HUVEC cells. Briefly, cells at 80-90% confluency will be infected with Ad hTM at various multiplicity of infection (MOI) in F12K medium without any supplements for 30 min at 37° C. The medium will then be removed and fresh growth medium will be added. The cells may optionally be washed with saline before the addition of the fresh medium. The cells will be incubated at 37° C. for 48-72 hours and analyzed for hTM expression. RT-PCR will be performed post infection using hTM specialized primers to detect for thrombomodulin mRNA. A hTM ELISA assay will be performed to determine hTM secretion in the culture medium. Western blots will also be performed to detect hTM protein expression in the virus infected cells.

As a control, the same cells will be infected with the gutless adenovirus expressing β-galactosidase (gutless Ad LacZ). The infected cells will subsequently be stained with X-gal using the β-galactosidase reporter gene staining kit from Sigma (Saint Louis, Mo.). Briefly, cells will be rinsed with PBS, fixed for 10 min at room temperature with the fixation solution, and then stained at 37° C. for 0.5-2 hours with the staining solution. The lacZ positive cells will be counted under a microscope. This will demonstrate the viability of the gutless adenovirus backbone itself.

### EXAMPLE 5

### In Vivo Expression of hTM

In vivo expression of hTM will be achieved by administering a therapeutically effective amount of the gutless Ad hTM into a rodent. The virus may administered intravascularly, subcutaneously, or intramuscularly. As is well-known to one skilled in the art, the dose and route of viral administration may vary based on the disease to be treated and the severity of the disease.

### EXAMPLE 6

### Ex Vivo Expression of hTM

Ex vivo expression of hTM will be achieved by introducing the hTM gene into cultured cells, such as human umbilical vein endothelium cells (HUVEC) cells, with viral or non-viral vectors. The hTM-expressing cells will then be implanted in a patient to provide a local supply of hTM. As is well-known to one skilled in the art, the dose and site of cell implantation may vary based on the disease to be treated and the severity of the disease. Similarly, hTM gene may be introduced into sections of blood vessels in vitro and the vessels will then be implanted in a patient.

### EXAMPLE 7

### Expression of hTM In Vitro Using Calcium Phosphate Precipitation

The hTM cDNA will be cloned into an expression vector under the control of a constitutive promoter, such as a CMV promoter. The resulting plasmid, pCMVhTM, will be transfected in vitro into HUVEC cells using calcium phosphate precipitation. The transfection may be performed with the Calcium Phosphate Transfection Kit from Invitrogen (Palo Alto, Calif.). Briefly, cells will be placed in 100 mm or 60 mm dishes at the required density. Incubate overnight at 37° C. in a humidified CO₂ incubator. 3-4 hours prior to transfection, the media will be changed on the dishes. A transfection mixture will be prepared by slowly add solution A dropwise to solution B while bubbling air through solution B with a pipette. The transfection mixture will be incubated at room temperature for 30 minutes, and will then be add dropwise to the media to the cells in either a 60 mm or 100 mm dish. The cells will be incubated with the transfection mixture at 37° C. for 30 min, washed with 1× Phosphate Buffered Saline (PBS), and incubated with fresh media at 37° C. for 48-72 hours. Optionally, a glycerol or DMSO shock may be carried out as described in the instruction manual to improve transfection efficiency. Expression of the hTM will be detected by analyzing the tissue culture medium using the IMUBIND® thrombomodulin ELISA kit from American Diagnostica Inc (Greenwich, Conn.). Alternatively, the cells will be harvested 48-72 hours post transfection. Western blots will be performed to confirm the hTM protein expression in the transfected cells.

### EXAMPLE 8

### Expression of hTM In Vitro Using Lipofectin Transfection

HUVEC cells will be transfected in vitro using a plasmid carrying the hTM gene and the Lipofectin reagent from Invitrogen. Briefly, HUVEC cells will be seeded in a six-well or 35-mm tissue culture plate in 2 ml of the appropriate growth medium supplemented with serum. The cells will be incubated at 37° C. in a CO₂ incubator until the cells are 40-60% confluent. On the day of transfection, the following solutions will be prepared in 12×75 mm sterile tubes:
Solution A: For each transfection, dilute 1-2 μg of DNA into 100 μl serum-free medium.
Solution B: For each transfection, dilute 2-20 μl of Lipofectin® Reagent into 100 μl serum-free medium, and allow to stand at room temperature for 30-45 min.
   The two solutions will then be mixed gently and incubate at room temperature for 10-15 min. The cells will be washed once with 2 ml of serum-free medium. For each transfection, 0.8 ml serum-free medium will be added to each tube containing the Lipofectin® Reagent-DNA complexes. The mixture will be added dropwise to the cells. The cells will be incubated for 30 min at 37° C. in a CO₂ incubator. The DNA-containing medium will then be replaced with 2 ml of normal growth medium containing serum. The cells will be incubated at 37° C. in a CO₂ incubator for a total of 48-72 hours. Expression of the hTM will be detected by analyzing the tissue culture medium at various time points post transfection using the IMUBIND® thrombomodulin ELISA kit from American Diagnostica Inc. Western blots will also be performed to confirm the hTM protein expression in the transfected cells.

### EXAMPLE 9

### Expression of hTM In Vitro Using NeoPhectin™ Transfection

The plasmid pCMVhTM will be transfected in vitro into HUVEC cells using the NeoPhectin™ reagent from NeoPharm (Lake Forest, Ill.). Briefly, appropriate number of cells will be incubated in 100 μl of culture medium containing 10% fetal bovine serum in a CO₂ incubator at 37° C. until the cells are 80-90% confluent. For each transfection per well, 1-7 μl of NeoPhectin™ will be mixed with 25 μl serum-free medium in a sterile tube and incubate at room temperature for 5 min. DNA (plasmid) in 25 μl serum-free medium will be combined with NeoPhectin™ dilution to a total volume of 50 μl in a sterile tube and incubated for 30 min to form lipid-nucleic complexes. The lipid-nucleic complexes will be added to each well containing cells and medium and incubated with the cells for 30 min at 37° C. in a CO₂ incubator. The medium will be replaced with 10% FBS media. Expression of the hTM will be detected by analyzing the tissue culture medium at 24-72 hours post transfection using the IMUBIND® thrombomodulin ELISA kit from American Diagnostic Inc. Alternatively, the cells will be harvested 48-72 hours post transfection. Western blots will be performed to confirm the hTM protein expression in the transfected cells.

### EXAMPLE 10

### Delivery hTM In Vitro Using Micro Emulsions

HUVEC cells will be transfected in vitro using a plasmid carrying the hTM gene and micro emulsions. Preparation of micro emulsions has been described, for example, by Yi et al. [Yi et al., Pharmaceutical Research, 17:314-320, (2000)], Cui et al. [Cui et al., Bioconjugate Chem. 13:1319-1327, (2002)], and in U.S. Pat. Nos. 5,061,688 and 5,438,041. Briefly, an emulsion/DNA complex will be prepared, added to cultured HUVEC cells, and incubated with the cells for 30 min. The medium will be replaced with 10% FBS media. Expression of the hTM will be detected by analyzing the tissue culture medium at 24-72 hours post transfection using the IMUBIND® thrombomodulin ELISA kit from American Diagnostic Inc. Alternatively, the cells will be harvested 48-72 hours post transfection. Western blots will be performed to confirm the hTM protein expression in the transfected cells.

### EXAMPLE 11

### Thrombomodulin ELISA

The amount of hTM in the tissue culture medium (in vitro expression) or in the plasma (in vivo expression) will be determined using IMUBIND® Thrombomodulin ELISA kit from American Diagnostica Inc. (Greenwich, Conn.). Briefly, samples will be diluted in Sample Buffer. 200 μl of the thrombomodulin standard, diluted reference plasma or diluted plasma sample will be added to the micro-test wells and incubated for 1 hour at room temperature. The wells will be washed 4 times with the Wash Buffer. 200 μl of Detection Antibody will be added to each well and incubated for 30 minutes at room temperature. The wells will be washed 4 times with the Wash Buffer. 200 μl of Substrate solution will be added to each well and incubated for 20 minutes at room temperature. A blue color will be developed, and the enzymatic reaction will be stopped by adding 100 μl of 0.5H₂SO₄. The solution color will turn yellow. The absorbances will be read on a micro-test plate reader at a wavelength of 450 nM within 30 minutes. The background average of the blanks will be deducted from the standards and sample readings.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1. A method for treating a thrombotic disease in a mammal, said method comprising:
administering to said mammal a therapeutically effective amount of a pharmaceutical composition comprising a viral vector,
wherein said viral vector comprises a nucleotide sequence encoding human thrombomodulin or its variant, and wherein said human thrombomodulin has an amino acid sequence recited in SEQ ID NO:2.

2. The method of claim 1, wherein said pharmaceutical composition further comprises a pharmaceutically acceptable carrier.

3. The method of claim 1, wherein said viral vector is an adenovirus.

4. The method of claim 3, wherein said adenovirus is a gutless adenovirus.

5. The method of claim 4, wherein said gutless adenovirus is produced using a shuttle vector comprising the nucleotide sequence recited in SEQ ID NO: 4.

6. The method of claim 1, wherein said nucleotide sequence encoding human thrombomodulin or its variant is operably linked to a constitutive promoter.

7. The method of claim 1, wherein said nucleotide sequence encoding human thrombomodulin or its variant is operably linked to a tissue-specific promoter.

8. The method of claim 1, wherein said nucleotide sequence encoding human thrombomodulin or its variant is under the control of a regulatable expression system.

9. The method of claim 1, wherein said thrombotic disease is atherosclerotic cardiovascular disease, pulmonary hypertension, acute inflammatory diseases, end-stage renal failure disease, or Alzheimer disease.

10. The method of claim 1, wherein said viral vector is an adeno-associated virus.

11. The method of claim 1, wherein said viral vector is a retrovirus.

12. The method of claim 1, wherein said viral vector is a lentivirus.

13. The method of claim 12, wherein said lentivirus is a human immunodeficiency virus.

14. The method of claim 1, wherein said viral vector is a herpes virus.

15. The method of claim 1, wherein said pharmaceutical composition is administered to said mammal intravascularly, subcutaneously, or intramuscularly.

16. A method for treating a thrombotic disease in a mammal, said method comprising:
administering to said mammal a therapeutically effective amount of a pharmaceutical composition comprising a non-viral vector, wherein said non-viral vector comprises a nucleotide sequence encoding human thrombomodulin or its variant, and wherein said human thrombomodulin has an amino acid sequence recited in SEQ ID NO:2.

17. The method of claim 16, wherein said pharmaceutical composition further comprises a pharmaceutically acceptable carrier.

18. The method of claim 16, wherein said non-viral vector is a liposome.

19. The method of claim 16, wherein said non-viral vector is a naked DNA molecule.

20. The method of claim 16, wherein the nucleotide sequence encoding human thrombomodulin or its variant is operably linked to a constitutive promoter.

21. The method of claim 16, wherein the nucleotide sequence encoding human thrombomodulin or its variant is operably linked to a tissue-specific promoter.

22. The method of claim 16, wherein the nucleotide sequence encoding human thrombomodulin or its variant is under the control of a regulatable expression system.

23. The method of claim 16, wherein said thrombotic disease is atherosclerotic cardiovascular disease, pulmonary hypertension, acute inflammatory diseases, end-stage renal failure disease, or Alzheimer disease.

24. A method for treating a thrombotic disease in a mammal, said method comprising:
administering to said mammal a therapeutically effective amount of thrombomodulin-producing cells,
wherein said thrombomodulin-producing cells are generated by introducing a polynucleotide encoding a human thrombomodulin or its variant into a cultured cell, and wherein said human thrombomodulin has an amino acids sequence recited in SEQ ID NO:2.

25. The method of claim 24, wherein said culture cell is human umbilical vein endothelium cell (HUVEC).

26. The method of claim 24, wherein said polynucleotide encoding a human thrombomodulin or its variant is introduced into said cultured cell by a viral vector.

27. The method of claim 24, wherein said polynucleotide encoding a human thrombomodulin or its variant is introduced into said cultured cell by a non-viral vector.

28. The method of claim 24, wherein said polynucleotide encoding a human thrombomodulin or its variant is introduced into said cultured cell by calcium phosphate precipitation.

29. The method of claim 24, wherein said polynucleotide encoding a human thrombomodulin or its variant is introduced into said cultured cell by electroporation.


SEQUENCE LISTING (6 sequences)

SEQ ID NO: 1 - DNA, 13599 bp, Artificial Sequence
  gutless backbone shuttle vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactggtaccgcggccgcctcgagtctagaactagtggatcccccgggctgcaggaattctgatggctctcaaaattcctgcctcctttagggataaaagactttaagactttttaacaaaaaagaaaaagaaaaaaaaaattcctgcctcctggtgtacacacacagaagggttccctccccttgaatgtgaccaggatctgtgaaaataacgggatagccgctcctgtgattaggttatgtggtagactagagcaagattctcctgctggttttgaagaagtcagctgccatgttgtgagactgtcatgggctagggcatgagcctttaaatatctgggagcaacccctggccagcagccagtgagaaaacgggccctcagtcctacaatcacaaggaactaaattctgccaacaacctgaaggaactttgaagaggatcatgagtcccttgattcagcttgatgagcccctgagcagaggatacagctaacttgtactagggaagtataaaaaacatgcatgggaatgatatatatcaactttaaggataattgtcatacttctgggaatgaagggaaagaaatggggctttagttgtattatgatctttaatttctcaaaaaaaataagatcagaagcaaatatggcaaaatgttaatacttttgtgggtacgtaggtattcagcatacccttttttctgagttcaaaatattttataattaaaatgaaatgcaggccaggcacagtggctcatgcctataataccagcactttgcgaggccgaggtgggaggatggcttgaggccagaccagcctggccaacatggcaaaaccccatctctacttaaaaaaaaaaaaactatatatatatatatgtgtgtgtgtgtgtatatatatatatgtatatatatttatatatgtgtgtatatatatatatgtatatatatttatatatgtgtgtgtatatatatatatacacacacacacatatatacatacatacatacacacacacacacacacaattagccaggcatggtggcgcacacctgtagtcccagctacttgggaggctgagacatgagaattgcttgaacctgggaggcagagtagttagtgagctgagatcataccactgcactccagcctggtgacagagtgagactctgtcttaaaaaaaataaaaattaaaattaaatgcaaaaggtccaagtgaattgaagaggaaaggggtatcaaggaggttttgtggaggtgacgtttgagctgggtcttaaatgacttaaacatgggataagaagggagggaataaggacattttcaggtacgagaaataaggagcaaacagtggaaacaacctaacgtctgtcaaccagtgaatggataacaaaaatgtaatcagatggtatccaacttacgatggttcaacatgagatttttctgactttaggatagatttatcaaagtagtaaatccattttcaacttatgatattttcaacttcagatgggtttatcaggacacagttgaggaacacctgtctatccatacaatttggcaataaaaaggaaatgagtgcagatatactccacaacatgaatgaaccttgaaaacattaagtgagagaagccagatacaaaaggccacatattgtatgattctatttatacaaaatgtccagaataggcaaatcttatagacagcaagtaggtagatgatcagtttgctaggtgctgggggaaggggaaatggggagtgatggctaaggggattgggtttctttgtggggcaatgaaaatgttttaaaattgagcgtgataatgattgcacaatgctgcatatatatataatctatagattatatatatataaagagaggctgttagacagtgataagtgatatatatatatatatacatagagagagagagagagagagagagaggctgttagtgataagtgatcaggaaaataaaagtattgaggaggaatacgaagttgacggtgtgaaaacatgagattttatataggatggccagggaaggccttaatgagaaagtgacttatgagtaaaaacaagggatcctaaaccttagcatgcatcagaatcactcggaaacttgttaaagcatagcttgctgggcctcatcacagatattttgattcggtaggttcttgtctgatattaatacttttggtctagggaaccacattttgagaaccactgagctaaaggaagtaaaggtttcccttagtttactagctggtaaccctaggaaactgcttagcctctcggtgctaagatacaaaatactttagcacataataacacatggaaaatagtctataaattataaatattattttttatgtaccaaatattacataagacaaaatctaagcaagatatatatatatatacataaaatataagatatatatgtatatattatatatagataaatagagagagagagttatgtttagaaagaaaatacttcaaactaaaaaaagagaggtaggaagtataccattccattattggtaaaaacaaattactaagtagtctttacaaaaaaccaatctcactcctttagaacacaagcccaccattaaaactgatgcagaggaatttctctccctggcttacctttaggatggtgcatactaagttagaaaagtcataaatgttatattaaaagtaaatgtgaacttacttccacaatcaagacattctagaagaaaaagagaaatgaaaatcagtacaatgaataaaacggtatttccaattataagtcaaatcacatcataacaaccctaaggaattatccaaactcttgtttttagatgctttattatatcaaactctcctttaaacaagtggcccatctgctgggatttggaagcctgtaatactgaaattttcatcataatggaaattttaaaaacagaatttgacccacctgtttttaaaacactttcattacttaacaagaggtctaatcttgggcaagtcttgaaatttctctggccttagtttccatgtgttaaatgaaacttgaagcagttggtctcttatagtctcctgactctaacattctaagaattatatttgtacaataactcaaaaatcacataatttaatttaccatatggactccaaaatatattttctcattaggctaaacttgatctgcattttctggatgtgtccatattcttggactacactaaaacatgataccaatgcttcctctcaccataaacccctcacttcgctttctacatttaagaattttatagctggaagagtccttaacagaaaataccatctaataattacccctcaaaatcgagaaagtcctatctgttcttatgctagttataagaatgaggcagcatttcacataatggttataaacactgccacaagaagattcatgatgtgttgtttatctgtagctctcatcatactctgtcatataactatagcattaagattttaatgttctatatattcttctaagacagtgtttaccagagtaaggcacaaaagatccactggtttgcaagaaagattagaacttttaaattttttacctcaccttgtttaatctatatttttgtatgtattttgtaacatatatattattattaccataaatcatatataatttaaaatgcatatattaggggtaaatgctcaggaaactttttataaattgggcatgcaaatacaagtttgaagactcactgttctaggtattaaaagtaaagttataaccaagtaaagcttccaccttttcatgtctcaaagcagtttattgttggaggtaagatctcttagaagcctaaacaggtccaagtacagaatgaagtaaggctagcccataacttgtggcaagcaattcatactatttctctcatgctgagctctcctcagtgaagcagctactatagacaactgcagcctattggtagcctattttacaggcaggaaaaaaattactttttattcaaagtggaactcaggacatggggagaaaatgaatacaaaaaataggtcaatccaaaggcacacagcaaatgagtaacacagttatgtttttttcccatttgtatgaggtcccagtaaattctaagtaaactgcaaatttaataatacactaaaaaagccatgcaattgttcaaatgaatcccagcatggtacaaggagtacagacactagagtctaaaaaacaaaagaatgccattattgagtttttgaattatatcaagtagttacatctctacttaataaatgagaaaaacgaggataagaggccatttgataaaatgaaaatagccaagaagtggtattagagacttgaatacaggtattcgggtccaaagttcatctgctcaaatactaactggggaaaagagggaaaaatatttatatacatatatatctgcacacaaaaatacccccaaaagacaaaatgaggccaggcagggtggctcacacccgtaatcccggtactttgggaggctgaggcaggtggatacctgagatcaggagttggagatcagcctggtcaacatggtgaaaccctgtctctactaaagataaaaaaattagccaggcatggtggcgtgcgcctgtaatcccagctacttgggagtctgaggcaggagaatcacttgaactgggaaggggaggttgcagtgagccaagatcgtactactgcactccagcctgggcagcagagtgagactccatcacaaaaataaataaataaataaaatacaatgaaacagaaagttcaaataatcccataatcttaccaccaagaaataactttcactcgttatacttattgatttttccataataaatgtactttactgtgactatcatgaaaagaaagttattttagaaacagagaactgtttcagatcaaatctatgtagtagaacagagccattaggtgggaaagacgagatcaaactaaatctcagaaggcctaaaaggctaggtccattccagcactaaaaactgaccagacaagtaatggcttcaacagcttctaaatatggacaaagcatgctgaaagggaaggacaggtctaacagtggtatatgaaatgaacaggaggggcaaagctcatttctcctctgaagttttccaaagatgctgaggaggacattagtttgacatgaccctgatatgggacaagataatttcacagaagttttacatgttaaagttttcttatagatactcattcaagtaagcaatgaacactaaaatctaaagaaagaaaagagctttagagtcaggtctgtattcaaattcaagctctaccacttactggttctgtgactttgggcaagtcttttaaccttattaagtcttaatttcctgatttgtaaaatggggatatcgtctccctcacaggattgttgtgaaacttttatgagattaatgcctttatatttggcatagtgtaagtaaacaataactggcagcttcaaaaaaaaaaagcagtagcattccatcatttattattggttactctcaaaaagtttttcaatgtactagaagataaatattcaaataccttaatatctccattattttcaggtaaacagcatgctcctgaacaaccaatgggtcaacaaataaattaaaagggaaatctaaaaacatcttgatattaaactacatggaagcacaatataccaaaaccaatggttcacactaggagaattttaaggtacaagaaaactctttgagatttcttaaaataatagtatgtctgaatttattgagtgatttaccagaaactgttgtaagagctctacttgcattatagcacttaatcctcttaactctatggctgctattatcaacctcaccctaatcacatatgggacacagagaggttaagtaacttgcccaaggtcagagttaggaagtactaagccatgctttgaatcagttgtcaggctccggaactcacactttcagccactacataatactgctttgctatcttttaggaaactatgtgagtctacctcacatagactcacataggtttgttttttttttttttttaaaggctatcttttcccccatcaatgttttttgaaggatcccaaattagagtcccacagaggcagacagcagtacttgacaatatggacatttaaggttaatgttggattctactgtctttttactacatgacctagggaacgataattaacctagactgcttccaagggttaaataacccatttagttatactatgtaaattatctcttagtgattgattgaaagcacactgttactaattgactcggtatgaagtgcttttttttcttccctttcaagatacatacctttccagttaaagttgagagatcatctccaccaattacttttatgtcccctgttgactggtcattctagttaaaaaaaaaaaaaactatatatatatatatctacacacacatatgtatatgtatatccttatgtacacacacaaacttcaaattaaatgagaactagaagatttgagaagttagctagctaatatccatagcattatgatattctaaatgatatgaattataagaattaggtttcctgaaatgaatgactagaaaactttcaagtagagattagtaaaaattaaaaagtcctaatcggccattactgatttgatgtttttaagagtcctaaaaaatgggttacatccatttttaagtgggtagtattataacagccacccatcttcaatcacagtgatttctgaattgtgagggaagttattagcatgacaggtgtctggttctggccctgtacgattcccatgagtcaagcaaattgtaagggctggtctatatcacacccaaccccaaggatatgtccctcaaaagtctagcccaggccccgtcatcttcagcatcatctgggaaaccaggtctgattagtagtcctttaaggaatacctcttaggctcccattttactgctatcacagaatccaataaaacccttacaggagattcaatgggaaatgctcaacacccactgtagttggtggtgacaatgaccataatttggctgtgctggattcaggacagaaaatttgggtgaaagagcaggtgaacaaaagagcttcgacttgccctagcagagagcaagccataccataccacaaagccacagcaattacaacggtgcagtaccagcacagtaaatgaacaaagtagagcccagaaacagacccagaactatatgaggatttagtatacaataaagatggtatttcgagtcagtagggaaaagatgaattattcaataaatgatgtttggccaactagtaacccatttgggaaaaaataaaagtatggtccctacctcacagcatacacaaaaataaattccagacggattaaaatctaaatgtaaaaaataaagccataagtggactggaagaaaatagagaattttttttaacatccgtagaaagggtaaaaacccaggcatgacatgaaccaaaactgaagaggttctgtaacaaatacccccttttatatattgggctccaacaataagaacccataggaaaatggagaatgaacacaaatagacaatttatagaagagaaggttataaggtgtaaaattatatctatctgagaaacaaacactaaaacaatgtgattctactgttctcccacccatactggcaaaacttaagcctgataatatgctgaggggaaataagcactcttgttggtgagagtattaattggcatagcttcttttgaaaatgacatagcaatacctgttaaaattgcaaacatgcatgtcacttaatccagtaatcccacttctgggaatcaatgctacaaaaacactgacaagtatacaaagatacattcaagagtgttcactgggccgggtgcggtggcttcatgcctgtaatcccagggaggcagaggcaagacgatcgcttgaccccaggagttcaaggccagcccgagaaacacagcaagaccctgtctctcttttttttatttaaaaaataaatgttcactgtatcagttgttcacaaaaacaaaccaacatgtccattaacagggaaccatttaaattaatcaagttcatctacacaatgtaataccatgcaactattaaaaagcacctgataatccaaagcacactgagacagaataatgctattaaaaacaccaagtagtggaacactgtgttgcctatgacaccatttttattcaacatttaaacaaatttgtaacagcaattacatgagtagtgacaatggcgtttatgagacttttcacttttatgtgcttctatttttgttatgcttctatatatacatccatttattatggagtgttactttcaaaaatcacaaatgggccagtattatttggtgttgcaaggtgagcatatgacttctgatatcaacctttgcatattacttctcaatttagggaaattacagacatcccttattctaactaacttaaaacccagcatttcaaacatacagaattgatggggaaaaaaaagaaagaagaaagaaagaaaaggcaacaagcttcagatgacagtgactcacatcaaattatttataaaatctgttaaatagtgccatcttctggagatacctggtattacagtccaactccagttgatgtctttacagagacaagaggaataaaggaaaaaatattcaagaactgaaaagtatggagtcatggaaaaattgctgtgatccaaaggctacggtgataggacaagaaacaagagaactccaagcagtaagacactgctgttctattagcatccaaacctccatactcctgtttgccccaaggcttttttaaaaaatagagacaggatctcactattttgctcaggctggtcttgaactcctggactcaagctatcctcctgcctcggcctcctaaagtgccgagattacaggcttgagtcaccatacctggctatttattttttcttaactctcttgcctggcctatagccaccatggaagctaataaagaatattaatttaagagtaatggtatagttcactacattggaatacaggtataagtgcctacattgtacatgaatggcatacatggatcaattaccccacctgggtggccaaaggaactgcgcgaacctccctccttggctgtctggaacaagcttcccactagatccctttactgagtgcctccctcatctttaattatggttaagtctaggataacaggactggcaaaggtgaggggaaagcttcctccagagttgctctaccctctcctctacgtcctatctcctcactcctctcagccaaggagtccaatctgtcctgaactcagagcgtcactgtcaactacataaaattgccagagaagctctttgggactacaaacacatacccttaatgtctttatttctattttgtctacctcttcagtctaggtgaaaaaataggaaggataatagggaagaactttgtttatgcctacttatccgcccctaggaattttgaaaacctctaggtagcaataagaactgcagcatggtatagaaaaagaggaggaaagctgtatagaaatgcataataaatgggcaggaaaagaactgcttggaacaaacagggaggttgaactataaggagagaaagcagagaggctaatcaacaaggctgggttcccaagagggcatgatgagactattactaaggtaggaattactaagggctccatgtccccttagtggcttagtactatgtagcttgctttctgcagtgaacttcagacccttcttttaggatcctagaatggactttttttttttatcggaaaacagtcattctctcaacattcaagcaggccccaagtctaccacactcaatcacattttctcttcatatcataatctctcaaccattctctgtccttttaactgtttttctataccctgatcaaatgccaacaaaagtgagaatgttagaatcatgtatttttagaggtagactgtatctcagataaaaaaaaagggcagatattccattttccaaaatatgtatgcagaaaaaataagtatgaaaggacatatgctcaggtaacaagttaatttgtttacttgtattttatgaattccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggatcgaattaattcttaattaa

SEQ ID NO: 2 - protein, 575 aa, Homo sapiens
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 3 - DNA, 1723 bp, Homo sapiens
atgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtacctcgagtgcatctgcgggcccgactcggcccttgcccgccacattggcaccgactgtgactccggcaaggtggacggtggcgacagcggctctggcgagccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactc

SEQ ID NO: 4 - DNA, 4457 bp, Homo sapiens
  misc_feature 349: n = A,T,C or G
gtttaaacgggccctctagacgcgttgacattgattattgactagttattaatagtaatcaattacggggtcattagttcatagcccatgatatcatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgcccccctattgacgtcaatgacggtaaatggcccgcctggcattatgcccagtncatgaccttatgggactttcctacttggcagacatctacgtattagtcatcgctattaccatggtgatgcggttttggcagtacatcaatgggcgtggatagcggtttgactcacggggattttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccctgcttactggcttatcgagatatctgcagaattcatctgtcgactgctaccggcagcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtgaaaaaaaaaaaaaaaaaaaaaaaaaaaaaaaaaaaaaaaaggtagcagtcgacagatgaattccaccacactggactagtggatccgagctcggtaccaagcttaagtttaaac

SEQ ID NO: 5 - DNA, 649 bp, Homo sapiens
  misc_feature 335: n = A,T,C or G
tctagacgcgttgacattgattattgactagttattaatagtaatcaattacggggtcattagttcatagcccatgatatcatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgcccccctattgacgtcaatgacggtaaatggcccgcctggcattatgcccagtncatgaccttatgggactttcctacttggcagacatctacgtattagtcatcgctattaccatggtgatgcggttttggcagtacatcaatgggcgtggatagcggtttgactcacggggattttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccctgcttactggcttatcgagatatc

SEQ ID NO: 6 - DNA, 3692 bp, Homo sapiens
ggcagcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtgaaaaaaaaaaaaaaaaaaaaaaaaaaaaaaaaaaaaaaaa